(19) United States
(12) Patent Application Publication
(10) Pub. No.: US 2023/0399707 A1
(43) Pub. Date: Dec. 14, 2023

(54) Methods, Compositions and Systems for Respiratory Virus Detection

(71) Applicant: Laboratory Corporation of America Holdings, Burlington, NC (US)

(72) Inventors: Ayla Harris, Stokesdale, NC (US); Brian Krueger, Burlington, NC (US)

(21) Appl. No.: 18/131,262

(22) Filed: Apr. 5, 2023

Related U.S. Application Data
(60) Provisional application No. 63/327,645, filed on Apr. 5, 2022.

(51) Int. Cl. C12Q 1/70

(52) U.S. Cl. CPC C12Q 1/701; C12Q 1/686

(57) ABSTRACT

Disclosed are methods, compositions and systems to measure respiratory viruses including, but not limited to, SARS-CoV-2, influenza A (Flu A), influenza B (Flu B), or Respiratory Syncytial Virus (RSV). The methods to determine whether a sample from a subject contains an infections respiratory virus may include the steps of obtaining a sample from a subject; performing RT-PCR using primers specific for at least two distinct respiratory viruses; and determining the amount and/or presence of at least one of the at least two distinct respiratory viruses in the sample. In certain cases, the methods and systems comprise multiplex real-time RT-PCR.

### RELATED APPLICATIONS

This application claims priority to U.S. Provisional Patent Application No. 63/327,645 filed Apr. 5, 2022. The disclosure of U.S. Provisional Patent Application No. 63/327,645 is incorporated by reference in its entirety herein.

### SEQUENCE LISTING

The instant application contains a Sequence Listing which has been submitted electronically in xml format and is hereby incorporated by reference in its entirety. The file with the sequence listing, created on Mar. 30, 2023, is named LC 2022-05-US (057618-1379609).xml and is 16,147 bytes in size.

### FIELD OF INVENTION

Disclosed are methods, compositions and systems for respiratory virus detection.

### INTRODUCTION

SARS-CoV-2 is an enveloped, single-stranded RNA virus of the family Coronaviridae, genus Beta coronavirus. All coronaviruses share similarities in the organization and expression of their genome, which encodes 16 nonstructural proteins and the 4 structural proteins: spike (S), envelope (E), membrane (M), and nucleocapsid (N). Viruses of this family are of zoonotic origin. They cause disease with symptoms ranging from those of a mild common cold to more severe ones such as the Severe Acute Respiratory Syndrome (SARS), Middle East Respiratory Syndrome (MERS) and Coronavirus Disease 2019 (COVID-19). The SARS-CoV-2 virus can cause a serious or life-threatening disease, including severe respiratory illness, to humans infected by this virus. On Feb. 11, 2020, the disease caused by SARS-CoV-2 was formally designated as Coronavirus Disease 2019 (COVID-19).

Influenza A causes flu in birds and some mammals, including humans. Influenza A is responsible for many cases of seasonal flu, which in the U.S., may result in over 30,000 deaths and as many as 200,000 hospitalizations each year. Human flu symptoms usually include fever, cough, sore throat, muscle aches, conjunctivitis and in severe cases may lead to pneumonia. Influenza type A viruses are categorized into subtypes based on the type of neuraminidase (NA) and hemagglutinin (HA) expressed by the viral genome. NA and HA are antigenic proteins present on the surface of the viral particles. Subtypes that spread widely among humans include H1N1, H1N2 and H1N2. Additionally, a highly pathogenic H5N1 avian influenza can infect humans with severe symptoms. Other variants known to infect humans have been described (e.g., H2N2, H3N2, H5N2, H5N8, H5N9, H7N3, H7N7, H7N9, H9N2, H10N7, H10N3).

Influenza B is the only species in the genus Betainfluenzavirus of the Orthomyxoviridae family and is known to infect humans, pigs and seals. Influenza viruses A and B are estimated to have diverged from a single ancestor around 4,000 years ago. While not as prevalent as Influenza A, Influenza B is still a significant health concern. For example, the flu FDA proposed flu vaccine for 2022-2023 included a combination of Influenza A and B like virus strains.

Respiratory Syncytial Virus (RSV) is a common contagious virus that causes infections of the respiratory tract. RSV is the most common cause of respiratory hospitalization in infants, and reinfection is common in later life. RSV can cause bronchitis, common colds and/or pneumonia. RSV is a negative sense single-stranded RNA virus.

Clinical signs and symptoms of respiratory viral infection due to SARS-CoV-2, influenza A, influenza B, or RSV can be similar to each other. With continued outbreaks of not only SARS-CoV-2 infections, but other respiratory viruses that were unusually scarce in the 2020-21 season, there is a need for tests that can detect and differentiate respiratory viruses.

### SUMMARY

Disclosed are methods, compositions and systems to detect infections by a respiratory virus (e.g., a Seasonal Respiratory Virus RT-PCR Test). The methods, compositions and systems may be embodied in a variety of ways.

In certain embodiments, disclosed is a method to determine whether a sample from a subject contains an infections respiratory virus comprising: obtaining the sample from a subject; isolating RNA from the sample; performing reverse transcriptase polymerase chain reaction (RT-PCR) amplification using primers specific for at least two distinct respiratory viruses; and determining the amount and/or presence and/or absence of at least one of the at least two distinct respiratory viruses in the sample.

Also disclosed are systems, as well as compositions and kits, for performing the disclosed methods. Additionally disclosed are computer-program products tangibly embodied in a non-transitory machine-readable storage medium, including instructions configured to run any of the stations/components of the system and/or to use any of the compositions and/or kits and/or or to perform any of the steps of the methods.

### BRIEF DESCRIPTION OF THE DRAWINGS

The disclosed method and systems may be better understood by reference to the following non-limiting figures.

FIG. 1 shows a method for the detection of at least two respiratory viruses in accordance with an embodiment of the disclosure.

FIG. 2 shows a system for the detection of at least two respiratory viruses in accordance with an embodiment of the disclosure.

FIG. 3 shows an exemplary computing device in accordance with various embodiments of the disclosure.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, method steps, or parts of a system, including circuits, systems, networks, processes, and other components may be shown as components in block diagram form in order not to obscure the embodiments in unnecessary detail.

### Definitions

The present disclosure now will be described more fully hereinafter. The disclosure may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section or as used elsewhere herein prevails over the definition that is incorporated herein by reference.

When introducing elements of the present disclosure or the embodiment(s) thereof, the articles “a”, “an”, “the” and “said” are intended to mean that there are one or more of the elements. The terms “comprising,” “including” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

Various aspects of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. Thus, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something.

“Sample” or “patient sample” or “biological sample” or “specimen” are used interchangeably herein. Samples may include upper and lower respiratory specimens. Such specimens (samples) may include anterior nasal samples (e.g., swabs), nasopharyngeal (NP) samples (e.g., swabs) or oropharyngeal samples (e.g., swabs), sputum, lower respiratory tract aspirates, bronchoalveolar lavage, and nasopharyngeal washes/aspirates or nasal aspirates. Other non-limiting examples of samples include a tissue sample (e.g., biopsies), blood or a blood product (e.g., serum, plasma, or the like as well as dried blood, serum or plasma), cell-free nucleic acids, urine, a liquid biopsy sample, or combinations thereof. The term “blood” encompasses whole blood, blood product or any fraction of blood, such as serum, plasma, buffy coat, or the like as conventionally defined. In certain embodiments, RNA is isolated from the sample. In certain embodiments, the samples may be self-collected by the subject.

As used herein, the term “subject” or “individual” refers to a human or any non-human animal. A subject or individual can be a patient, which refers to a human presenting to a medical provider for diagnosis or treatment of a disease, and in some cases, wherein the disease may be any infection by a pathogen.

As used herein, a “pathogen-specific nucleic acid” or “pathogen nucleic acid” is a nucleic acid molecule that is not normally present in the subject but is a sequence found in the pathogen genome. For example, a “SARS-CoV-2 specific nucleic acid” or “SARS-CoV nucleic acid” is not normally found in the human genome (or in samples from a human subject) but is a sequence derived from the SARS-CoV-2 genome. Similarly, influenza A, influenza B and RSV nucleic acids are pathogen-specific nucleic acids.

As used herein, “SARS-CoV-2” or “SARS-CoV2” or the “SARS-CoV-2 or SARS-CoV2 virus” includes all genetic variants of the virus including those that can cause the disease of COVID-19.

As used herein, “influenza A virus” includes all genetic variants of the virus including those that can cause the disease influenza A or Flu A. Influenza A is virus a negative-sense, single-stranded, segmented RNA virus. The several subtypes are labeled according to an H number (for the type of hemagglutinin and a N number (for the type of neuraminidase). There are 18 different known H antigens (H1 to H18) and 11 different known N antigens (N1 to N11). For example, H17N10 was isolated in 2012 and H18N11 was discovered in 2013.

As used herein, “influenza B virus” includes all genetic variants of the virus including those that can cause the disease influenza B or Flu B. Influenza B virus is the only species in the genus Betainfluenzavirus in the virus family Orthomyxoiridae, a family of negative-sense RNA viruses. There are two known circulating lineages of Influenza B virus based on the antigenic properties of the surface glycoprotein hemagglutinin. The lineages are termed B/Yamagata-like and B/Victoria-like viruses. Influenza B virus, like influenza A, has a linear, negative-sense single-stranded RNA segmented genome.

As used herein, “Respiratory Syncytial Virus (RSV)” includes all genetic variants of the virus including those that can cause disease. RSV causes infections in the respiratory system and is the single most common cause of respiratory hospitalization in infants. RSV has a negative-sense, single-stranded RNA genome. The genome is linear and not segmented.

As used herein, the term “nucleic acid” refers to a polynucleotide such as deoxyribonucleic acid (DNA) or ribonucleic acid (RNA). The term is used to include single-stranded nucleic acids, double-stranded nucleic acids, mRNA, and RNA and DNA made from nucleotide or nucleoside analogues.

As used herein, “copy DNA or cDNA” refers to DNA that is created from RNA using reverse transcriptase.

As used herein, RT-PCR refers to reverse transcription polymerase chain reaction. Thus, as used herein RT-PCR comprises reverse transcription of RNA into cDNA, followed by PCR amplification of the cDNA. Also, as used herein real-time RT-PCR or quantitative RT-PCR is used to measure the amount of the specific RNA that is amplified during PCR. This can be achieved by monitoring the amplification reaction using fluorescence. For example, real-time RT-PCR may comprise the step of amplifying a specific cDNA target sequence by hybridizing a probe to the specific target sequence such that during the extension phase of amplification a 5′→3′ nuclease activity of Taq polymerase degrades the bound probe causing a reporter dye on the probe to separate from a quencher dye on the thereby generating a fluorescent signal.

As used herein, a “cycle threshold” or “Ct” value refers to the number of amplification cycles required to create enough copies of a template to be detected.

As used herein, “TCID₅₀” or “Median Tissue Culture Infectious Dose” is the dilution of a virus required to infect 50% of a given cell culture.

As used herein, a “detectable moiety” is a chemical moiety that allows for molecule that is attached to be quantitatively measured. In certain embodiments, certain molecules (e.g., nucleic acid probes) used in accordance with and/or provided by the invention comprise one or more detectable entities or moieties, i.e., such molecules are “labeled” with such entities or moieties. Any of a wide variety of detectable agents can be used in the practice of the disclosure. Suitable detectable agents include, but are not limited to, various ligands, radionucleotides, fluorescent dyes, chemiluminescent agents (such as, for example, acridinium esters, stabilized dioxetanes, and the like), bioluminescent agents, spectrally resolvable inorganic fluorescent semiconductors nanocrystals (i.e., quantum dots), microparticles, metal nanoparticles (e.g., gold, silver, copper, platinum), nanoclusters, paramagnetic metal ions, enzymes, colorimetric labels (e.g., dyes, colloidal gold, and the like), biotin, digoxigenin, haptens, and proteins for which antisera or monoclonal antibodies are available.

In certain embodiments, a detectable moiety is a fluorescent dye. Numerous known fluorescent dyes of a wide variety of chemical structures and physical characteristics are suitable for use in the practice of the disclosure. A fluorescent detectable moiety can be stimulated by a laser with the emitted light captured by a detector. The detector can be a charge-coupled device (CCD) or a confocal microscope, which records its intensity.

Suitable fluorescent dyes used as reporter dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM), Yakima Yellow (YakYel), Texas Red (TexRd), AATTO 550 (ATT0550), and/or CY-5 (i.e., Cy5). Other suitable reporter dyes include hexachloro-fluorescein (HEX), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, or tetramethylrhodamine (TMR)), coumarin and coumarin dyes (e.g., methoxycoumarin, dialkylaminocoumarin, hydroxycoumarin or aminomethylcoumarin (AMCA)), Q-DOTS, Oregon Green Dyes (e.g., Oregon Green 488, Oregon Green 500 or Oregon Green 514), Texas Red-X, SPECTRUM RED, SPECTRUM GREEN, cyanine dyes (e.g., CY-3, CY-3.5, or CY5.5), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660 or ALEXA FLUOR 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650 or BODIPY 650/665), IRDyes (e.g., IRD40, IRD 700, or IRD 800), and the like. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as in fluorescent resonance energy transfer (FRET). Resonance transfer results an overall enhancement of the emission intensity. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer and/or probe. Donor/acceptor pairs of dyes that can be used include, for example, fluorescein/tetramethylrohdamine, IAEDANS/fluorescein, EDANS/DABCYL, fluorescein/fluorescein, BODIPY FL/BODIPY FL, and Fluorescein/QSY 7 dye. Many of these dyes also are commercially available, for instance, from Molecular Probes Inc. (Eugene, Oregon). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

Additionally, suitable fluorescent quencher molecules may be used. As used herein, fluorescent quenching refers to any process that decreases the fluorescence of a molecule such as black hole quenchers commercially available from Biosearch Technologies. Such quenchers include, but are not limited to, the double quenchers: ZEN™ in combination with an Iowa Black quencher (e.g., IABKFQ), and TAO™ in combination with an Iowa Black quencher (e.g., IABKFQSp). Or black hole quenchers BHQ0, BHQ1, BHQ3, and BHQ4 may be used. Different quencher dyes are suitable for use with specific fluorophores, including FAM, TET, JOE, HEX, Oregon Green®, TAMRA, ROX, Cyanine-3, Cyanine-3.5, Cyanine-5 and Cyanine-5.5 (e.g., CY-3, CY-5, CY-3.5, CY5.5).

In certain embodiments, a detectable moiety is an enzyme. Examples of suitable enzymes include, but are not limited to, those used in an enzyme-linked immunosorbent assay (ELISA), e.g., horseradish peroxidase, beta-galactosidase, luciferase, alkaline phosphatase. Other examples include beta-glucuronidase, beta-D-glucosidase, urease, glucose oxidase, etc. An enzyme may be conjugated to a molecule using a linker group such as a carbodiimide, a diisocyanate, a glutaraldehyde, and the like.

In certain embodiments, a detectable moiety is a radioactive isotope. For example, a molecule may be isotopically labeled (i.e., may contain one or more atoms that have been replaced by an atom having an atomic mass or mass number different from the atomic mass or mass number usually found in nature) or an isotope may be attached to the molecule. Non-limiting examples of isotopes that can be incorporated into molecules include isotopes of hydrogen, carbon, fluorine, phosphorous, copper, gallium, yttrium, technetium, indium, iodine, rhenium, thallium, bismuth, astatine, samarium, and lutetium (e.g., 3H, 13C, 14C, 18F, 19F, 32P, 35S, 64Cu, 67Cu, 67Ga, 90Y, 99mTc, 111In, 125I, 123I, 129I, 131I, 135I, 186Re, 187Re, 201Tl, 212Bi, 213Bi, 211At, 153Sm, or 177Lu).

### Methods to Detect Respiratory Virus Infection

Disclosed are methods to detect infections by a respiratory virus (e.g., a Seasonal Respiratory Virus RT-PCR Test). The method may be embodied in a variety of ways.

For example, in certain embodiments, disclosed is a method to determine whether a sample from a subject contains an infections respiratory virus comprising: obtaining the sample from a subject; isolating RNA from the sample; performing RT-PCR using primers specific for at least two distinct respiratory viruses; and determining the amount or the absence and/or presence of at least one of the at least two distinct respiratory viruses in the sample. In an embodiment, the RT-PCR comprises generating cDNA specific to the at least two distinct respiratory viruses. In certain embodiments, the method comprises real-time (i.e., quantitative) RT-PCR amplification. In certain embodiments, the method comprises multiplex RT-PCR. In certain embodiments, the at least two distinct respiratory viruses comprise at least two, or at least three, or all four of SARS-CoV-2, influenza A (Flu A), influenza B (Flu B), or Respiratory Syncytial Virus (RSV). Or other viruses may be detected.

Clinical signs and symptoms of respiratory viral infection due to SARS-CoV-2, influenza A, influenza B, or RSV can be similar to each other. Thus, in certain embodiments, the assay is used to determine if the subject is infected with a respiratory virus and to identify the respiratory virus. RNA from Influenza A, Influenza B, RSV, and SARS-CoV-2 can be detected in respiratory specimens during the acute phase of infection. Thus, in certain embodiments, positive results are indicative of active infection. In certain embodiments, clinical correlation with patient history and other diagnostic information may be used to determine patient infection status as in certain cases, positive results may not rule out bacterial infection or co-infection with other pathogens not detected by the test.

A variety of sample types may be used. In certain embodiments, the sample comprises a specimen from either the upper or lower respiratory system. For example, the sample may be an anterior nasal swab, a nasopharyngeal or oropharyngeal swab, sputum, a lower respiratory tract aspirate, a bronchoalveolar lavage sample, or a nasopharyngeal wash/aspirate or nasal aspirate. In certain embodiments, the sample is an anterior nasal swab or a nasopharyngeal swab. Or other types of samples may be used.

In certain embodiments, the sample may be self-collected by the subject. Thus, in certain embodiments, samples may be individual anterior nasal swab specimens that are self-collected by individuals using a Labcorp COVID-19+Flu+RSV Test Home Collection Kit (Rx) and/or a Pixel by Labcorp COVID-19+Flu+RSV Test Home Collection Kit (DTC). Or other types of self-collection kits may be used.

In certain embodiments, the primers used for RT-PCR amplify the SARS-CoV-2 nucleocapsid (N) gene nucleic acid. Additionally, and/or alternatively, the primers used for RT-PCR amplify the influenza A virus matrix 1 (M1) gene nucleic acid. Additionally, and/or alternatively the primers used for RT-PCR amplify the influenza B virus nonstructural 2 (NS2) gene nucleic acid. Additionally, and/or alternatively, the primers used for RT-PCR amplify the Respiratory Syncytial Virus matrix (M) gene nucleic acid. Or other targets for each of these viruses may be used.

In certain embodiments, the step of detecting further comprises amplification of a control gene that is present in the subject, but not the pathogen. The internal control may be used to determine whether any of the steps of sample isolation, RNA isolation, RT-PCR and or analysis have been performed according to predetermined standards. For example, the control gene may be the human RNase P (RP) gene or another human gene such as a housekeeping gene involved in basic cell maintenance. Or other control genes may be used.

In certain embodiments, the primers and probes shown in Table 1 below are used. Or other primers and probes may be used.

TABLE 1

| Target | Primer/Probe | Sequence 5′ to 3′ | Region | SEQ ID NO |
|---|---|---|---|---|
| Influenza | InfA For1 | CAA GAC CAA TCY TGT CAC CTC TGA C | M1 | 1 |
| A | InfA For2 | CAA GAC CAA TYC TGT CAC CTY TGA C | | 2 |
| | InfA Rev1 | GCA TTY TGG ACA AAV CGT CTA CG | | 3 |
| | InfA Rev2 | GCA TTT TGG ATA AAG CGT CTA CG | | 4 |
| | InfA Probe | /FAM/-TGC AGT CCT /ZEN/ CGC TCA CTG | | 5 |
| | | GGC ACG-/IABkFQ/ | | |
| Influenza B | InfB For | TCC TCA AYT CAC TCT TCG AGC G | NS2 | 6 |
| | InfB Rev | CGG TGC TCT TGA CCA AAT TGG | | 7 |
| | InfB Probe | /YakYel/-CCA ATT CGA /ZEN/ GCA GCT | | 8 |
| | | GAA ACT GCG GTG-/IABkFQ/ | | |
| RSV | RSV-MF | GGC AAA TAT GGA AAC ATA CGT GAA | M | 9 |
| | RSV-MR | TCT TTT TCT AGG ACA TTG TAY TGA | | 10 |
| | | ACA G | | |
| | RSV-Probe | /ATTO550/-CTG TGT ATG /TAO/ TGG AGC | | 11 |
| | | CTT CGT GAA GCT-/IAbRQSp/ | | |
| SARS- | SC2 For | CTG CAG ATT TGG ATG ATT TCT CC | N | 12 |
| CoV-2 | SC2 Rev | CCT TGT GTG GTC TGC ATG AGT TTA G | | 13 |
| | SC2 Probe | /TexRd/-ATT GCA ACA /TAO/ ATC CAT | | 14 |
| | | GAG CAG TGC TGA CTC-/IAbRQSp/ | | |
| Rnase P | RnaseP For | AGA TTT GGA CCT GCG AGC G | RPP30 | 15 |
| | RnaseP Rev | GAG CGG CTG TCT CCA CAA GT | | 16 |
| | RnaseP Probe | /Cy5/-TTC TGA CCT /TAO/ GAA GGC TCT | | 17 |
| | | GCG CG-/IAbRQSp/ | | |

Thus, in certain embodiments the primers for RT-PCR amplification of the Influenza A M1 gene sequences comprise SEQ ID NOs: 1, 2, 3 or 4 and/or the probe used for detection of amplification of the Influenza A M1 gene sequences comprises SEQ ID NO: 5. The use of two primer sets may provide for increased genome coverage. Additionally and/or alternatively, the primers for RT-PCR amplification of the Influenza B NS2 gene sequences may comprise SEQ ID NOs: 6 and 7 and/or the probe used for detection of amplification of the Influenza B NS2 gene sequences may comprise SEQ ID NO: 8. Additionally and/or alternatively, the primers for RT-PCR amplification of the RSV M gene sequences may comprise SEQ ID NOs: 9 and 10 and/or the probe used for detection of amplification of the RSV M gene sequences may comprise SEQ ID NO: 11. Additionally and/or alternatively, the primers for RT-PCR amplification of the SARS-CoV-2 N gene sequences may comprise SEQ ID NOs: 12 and 13 and/or the probe used for detection of amplification of SARS-CoV-2 N gene sequences may comprise SEQ ID NO: 14. Additionally and/or alternatively, the primers for RT-PCR amplification of RNase P gene sequences may comprise SEQ ID NOs: 15 and 16 and/or the probe used for detection of amplification of RNase P gene sequences may comprises SEQ ID NO: 17.

In some embodiments, the method comprises multiplex RT-PCR. Thus, in certain embodiments, cDNA (or RNA for one-step RT-PCR) from a single sample, may be combined with primers and probes for at least two, or at least three, or all four of SARS-CoV-2, influenza A (Flu A), influenza B (Flu B), or Respiratory Syncytial Virus (RSV). The multiplex PCR may, in certain embodiments, comprise two different primers for Influenza A (Table 1) and any of the other viruses, and/or an internal control, such as RNase P (Table 1).

In certain embodiments, assay targets may be reported as detected (+) if they produce a Ct value of ≤40 or not detected (−) if they produce a Ct value of >40. Such analysis may comprise determining whether expected results are obtained for the internal control (e.g., human RNase P), a positive template control (e.g., nucleic acid sequences specific to at least one of SARS-CoV-2, Influenza A, Influenza B and/or RSV), a negative extraction control (e.g., a sample from an individual not infected with any of the viruses being tested for) and/or a no template control (e.g., water or buffer). In certain embodiments, tests that are negative for all targets are interpreted to be valid if the RNase P internal control has a Ct value of ≤40. In certain embodiments, any test resulting as invalid is repeated at least once.

Examples of potential results and corresponding result interpretation are shown in Table 5. In certain embodiments, the limit of detection (LoD) for each of SARS-CoV-2, Influenza A, Influenza B and/or RSV is <10 copies per microliter (μL). Example LoD results are shown in Tables 8 and 9. In certain embodiments, the positive predictive agreement (PPA) for Flu A, Flu B, and RSV is 100%, and the negative predictive agreement (NPA) is 100%. In certain embodiments, the PPA for SARS-CoV-2 is 96.7% with a lower bound 95% confidence interval of 90.9%, and the NPA is 100%.

In some embodiments, the sample is heated to inactivate the pathogen. In alternate embodiments, the sample is heated to at least 60 degrees C., or to at least 65 degrees C., or to at least 70 degrees C., or to at least 75 degrees C. for a designated time. The sample may be heated for at least 10 minutes, or at least 20 minutes, or at least 30 minutes, or at least 40 minutes or at least 50 minutes or for 1 hour or more. In an embodiment, the sample may be heated at 65 degrees C. for about 30 minutes. In certain embodiments, the sample is treated with a protease to inactivate any virus in the sample. In an embodiment, a protease, e.g., proteinase K is also added to the samples prior to heat-inactivation. Or another protease may be used.

In some embodiments, for the analysis of viral RNA, the step of isolating viral RNA comprises nucleic acid extraction. A variety of methods may be used for extraction of nucleic acid from the sample. In an embodiment, the method may employ a semi-automated sample extraction protocol, as for example, a ThermoFisher Scientific KingFisher Flex Magnetic Particle processor.

Additionally, and/or alternatively, the samples may be subjected to methods to first concentrate the pathogen. For example, for isolation of viral particles, the samples may be subjected to concentration (e.g., purification) of the virus using a matrix designed to bind viral particles (e.g., Nanotrap® Virus Capture Kit (Ceres Nanosciences, Inc.). Using such a matrix, elution of viral RNA from the concentrated viral particles may be performed at a temperature of about 90-99 degrees C., for at least 3 minutes. In an embodiment, elution may be performed at 95 degrees C. for at least 5 minutes. The nucleic acid (e.g., RNA or DNA) may then be isolated from the sample.

Or other methods of purification may be used. For example, nucleic acid may be isolated using a protease (e.g., proteinase K) in an extraction buffer (e.g., HEPES buffer), EDTA, and a detergent (e.g., lithium lauryl sulfate) with or without added non-pathogen DNA (e.g., salmon sperm) and incubating at about 60-65 degrees C. for about 1 hour, followed by extraction in phenol-chloroform-isoamyl alcohol and ethanol precipitation. Or extraction in the presence of guanidinium isothiocyanate or other chaotropic agents may be performed.

Once the nucleic acid is extracted, samples may be set up for generation of cDNA and RT-PCR using an automated system. For example, the Hamilton Microlab STAR liquid handling system, or other automated handling systems, may be used. In an embodiment, one-step RT-PCR is performed.

In certain embodiments, after generation of cDNA amplification of viral specific sequences is performed. In an embodiment, the cDNA is specific to the virus of interest (e.g., SARS-CoV-2, influenza A (Flu A), influenza B (Flu B), or Respiratory Syncytial Virus (RSV). In an embodiment, the RT-PCR is real-time RT-PCR. Real-time RT-PCR may comprise the step of amplifying at least one specific target sequence for at least two of the viruses (or the internal control) by hybridizing a probe to the at least one specific target sequence such that during the extension phase of the amplification a 5′→3′ nuclease activity of Taq polymerase degrades the bound probe causing a reporter dye on the probe to separate from a quencher dye on the probe thereby generating a fluorescent signal. As discussed herein, for SARS-CoV-2 the target may comprise a portion of the nucleocapsid (N) gene. For Influenza A, the target may comprise a portion of the matrix 1 (M1) gene. For Influenza B, the target may comprise a portion of the nonstructural protein (NS) gene. For RSV, the target may comprise a portion of the matrix (M) gene. Thus, for detection of SARS-CoV-2, Influenza A, Influenza B and/or RSV, the step of amplifying at least one target sequence of SARS-CoV-2 may comprise hybridizing a probe to the at least one specific target sequence of the viral cDNA such that during the extension phase of the amplification the 5′ nuclease activity of Taq polymerase degrades the bound probe causing a reporter dye on the probe to separate from a quencher dye on the probe to generate a fluorescent signal. A variety of reporter and/or quenching dyes known in the art may be used. In certain embodiments, the reporter dye is FAM, YakYel, TexRed, or Cy5. Additionally, and/or alternatively, the quencher dye may be the double quenchers: ZEN™ in combination with an Iowa Black quencher (e.g., IABKFQ), and TAO™ in combination with an Iowa Black quencher (e.g., IABKFQSp) (see e.g., Table 1).

For quantitative PCR, the fluorescence intensity may then be monitored throughout amplification, e.g., at each PCR cycle or at select time points. For example, fluorescence intensity may be monitored at each PCR cycle. The methods may be automated. For example, in certain embodiments, an Applied Biosystems QuantStudio7 Flex (QS7) instrument with software version 1.3 may be used to monitor fluorescence intensity during the PCR amplification. Or other instruments and computer software for monitoring quantitative PCR may be used.

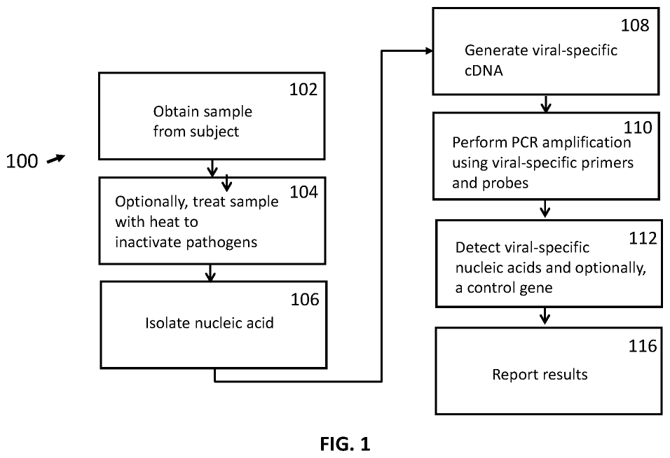

An embodiment of a method (100) of the disclosure is illustrated in FIG. 1. Thus, in an embodiment, a sample is obtained from a subject (102). In certain embodiments, the sample may be an anterior nasal, nasopharyngeal or oropharyngeal swab, sputum, a lower respiratory tract aspirate, a bronchoalveolar lavage sample, or a nasopharyngeal wash/aspirate or nasal aspirate. In certain embodiments, the sample is an anterior nasal or nasopharyngeal swab. Or other types of samples may be used.

The sample may be self-collected by the subject. For example, in certain embodiments, a Pixel by Labcorp COVID-19+Flu+RSV Test Home Collection Kit is available direct-to-consumer through the Pixel by Labcorp website. Individuals (i.e., the subject or patient) may be asked screening questions using a questionnaire on a Labcorp Pixel website when ordering the kit. A lab order may be generated by the Physician Wellness Network (PWN) and the individual may perform the sample collection and mail the sample back for testing.

Optionally, the sample may be treated with heat to inactivate pathogens present in the sample (104). In alternate embodiments, the sample is heated to at least 60 degrees C., or to at least 65 degrees C., or to at least 70 degrees C. or to at least 75 degrees C. for a designated time. The sample may be heated for at least 10 minutes, or at least 20 minutes, or at least 30 minutes, or at least 40 minutes, or at least 50 minutes or for 1 hour or more. In an embodiment, the sample may be heated at 65 degrees C. for about 30 minutes. In an embodiment, a protease, e.g., proteinase K or another protease, may be added to inactivate the pathogen. In certain embodiments, the protease is also added to the samples prior to addition of heat for heat-inactivation.

Also optionally, any pathogens present in the sample may be partially purified (e.g., concentrated) from the rest of the sample (not shown in FIG. 1). For example, for isolation of viral particles, the samples may be subjected to concentration (e.g., purification) of the virus using a matrix designed to bind viral particles (e.g., Nanotrap® Virus Capture Kit, Ceres Nanosciences, Inc.). Or other methods of purification may be used.

Next, the sample is processed to isolate nucleic acids from the sample (106). For example, in one embodiment, the method may employ a semi-automated sample extraction protocol as for example, a ThermoFisher Scientific KingFisher Flex Magnetic Particle processor.

At this point the nucleic acid may be processed to generate cDNA (108) and the presence and/or amount of viral nucleic acid may be determined by amplification using viral-specific primers and probes (110). In an embodiment, the generation of cDNA and subsequent amplification are performed as one-step RT-PCR. For detection of pathogen nucleic acid, sequences specific to the viruses (i.e., a plurality of SARS-Cov-2, Influenza A, Influenza B and/or RSV) may be amplified for subsequent detection. For example, in one embodiment, quantitative (i.e., real-time) PCR amplification, using primers specific to nucleic acid sequences in the pathogen and an internal probe may be used. In certain embodiments, multiplex RT-PCR is performed. In an embodiment, multiplex real-time RT-PCR is performed. In certain embodiments, the RT-PCR plates are set-up using a Hamilton Microlab STAR liquid handling system and amplification/detection is performed with the Applied Biosystems QuantStudio7 Flex (software version 1.3). In an embodiment, the internal probe may be labeled with a reporter and a quencher dye such that amplification allows the 5′→3′ exonuclease activity of Taq polymerase to release the reporter dye, thereby allowing amplification to be monitored. Any reporter and quencher dyes in the art may be used. In certain embodiments, the reporter dye is FAM, YakYel, TexRed, or Cy5. Additionally, and/or alternatively, the quencher dye may be the double quenchers ZEN™ in combination with an Iowa Black quencher (e.g., IABKFQ), and/or TAO™ in combination with an Iowa Black quencher (e.g., IABKFQSp) (see e.g., Table 1). In certain embodiments, the step of detecting further comprises amplification of a control gene that is present in the subject, but not the virus. For example, the control gene may be the human RNase P (RP) gene or another gene such as a housekeeping gene.

At this point, the RT-PCR results may be interpreted (112), for example, by analysis of the results of the multiplex real-time RT-PCR. Such analysis may comprise review of the data to determine the level of detection for at least one of SARS-CoV-2, Influenza A, Influenza B and/or RSV. In an embodiment, the level of detection is measured by a cycle threshold (Ct) value. In an embodiment, assay targets will be reported as detected (+) if they produce a Ct value of ≤40 or not detected (−) if they produce a Ct value of >40. Such analysis may comprise determining whether expected results are obtained for the internal control (e.g., human RNase P), a positive template control (e.g., nucleic acid sequences specific to at least one of SARS-CoV-2, Influenza A, Influenza B and/or RSV), a negative extraction control (e.g., a sample from an individual not infected with any of the viruses being tested for) and/or a no template control (e.g., water or buffer). In certain embodiments, tests that are negative for all targets are interpreted as valid only if the RNase P internal control has a Ct value of ≤40.

Next, results may be reported to the subject, or his or her health care provider, or other medical professional (114). For example, in an embodiment, test results are delivered to the user via their Pixel by Labcorp account. A Physician Wellness Network (PWN) may follow up positive and indeterminate or invalid test results by contacting the subject.

The methods can detect a plurality of respiratory virus isolates and/or strains. For example, for Influenza A, the method may detect A/California/07/09 (H1N1), A/Canada/6294/09 (H1N1), A/HongKong/4801/14 (H3N2), A/Mexico/4108/09 (H1N1), A/Michigan/45/15 (H1N1), A/Perth/16/09 (H3N2), A/Perth/16/09 (H3N2), A/Singapore/63/04 (H1N1), A/Switzerland/9715293/13 (H3N2), A/Texas/50/12 (H3N2), and/or A/Wisconsin/67/05 (H3N2). For influenza B, the method may detect B/Alabama/2/17 (Victoria), B/Brisbane/60/2008 (Victoria), B/Florida/78/2015 (Victoria), B/Washington/02/2019 (Victoria), B/Wisconsin/1/2010 (Yamagata), and/or B/Utah/9/2014 (Yamagata). For RSV, the method may detect RSV A 3/2015 Isolate #3, RSV A 2014 Isolate 341, RSV A 2013 Isolate, RSV A 2006 Isolate, RSV B CH93(18)-18, RSV B 3/2015 Isolate #1, and/or RSV B WV/14617/85. For SARS-CoV-2, the method may detect SARS-CoV-2 (Hong Kong/VM20001061/2020), SARS-CoV-2 (Isolate: Italy-INMI1), SARS-CoV-2 Variant B.1.351 SA, and/or SARS-CoV-2 Variant B.1.1.7 ENG. See e.g., Table 12. Or other strains and/or isolates may be detected.

The method may be automated. For example, in certain embodiments, the steps of treating the sample to isolate nucleic acids, performing RT-PCR, interpreting the results and/or reporting the results are performed in an automated fashion.

The method may be optimized to produce results in less than 1 day. For example, for the analysis of respiratory viruses as disclosed herein, the method may take less than 10 hours, or less than 8 hours, or less than 6 hours or less than 4 hours, or less than 3 hours, or less than 2 hours, or less than one hour. Also, as noted above, in an embodiment, the samples may be subjected to viral concentration and/or heat inactivation, and/or protease treatment prior to extraction of the viral nucleic acids. This can improve throughput, allow processing of multiple samples (e.g., 400 samples) in about 40 minutes or less. For example, in certain embodiments, the disclosed methods may be performed robotically. Robotic processing may allow for a large number of samples to be analyzed with a shorter turn-around than a completely manually performed method. In a robotic embodiment, samples may be robotically extracted from sample collection apparatus (e.g., a tube, vial, sample carrier etc.) for further analysis via any of the methods disclosed herein. In a further robotic embodiment, extracted samples may be placed in reaction vessel (e.g., tube, vial, etc.) for a PCR reaction according to any of the disclosed methods. Additionally, and/or alternatively, the PCR reaction may be performed robotically

Thus, an embodiment of the disclosed SARS-CoV-2, Influenza A, Influenza B and/or RSV RT-PCR test comprises a multiplex, real-time reverse transcription polymerase chain reaction (RT-PCR) test for the qualitative detection of nucleic acid from at least two of SARS-CoV-2, Influenza A, Influenza B and/or RSV in upper and lower respiratory specimens (such as anterior nasal, nasopharyngeal or oropharyngeal swabs, or sputum), lower respiratory tract aspirates, bronchoalveolar lavage, and nasopharyngeal wash/aspirate or nasal aspirate collected from individuals suspected of being infected with at least one of SARS-CoV-2, Influenza A, Influenza B and/or RSV.

In an embodiment, results are presented for the identification of SARS-CoV-2, Influenza A, Influenza B and/or RSV RNA. SARS-CoV-2, Influenza A, Influenza B and/or RSV RNA is generally detectable in respiratory specimens during the acute phase of infection. Positive results are indicative of the presence of the virus RNA detected. In some embodiments, clinical correlation with patient history and other diagnostic information may be used to determine patient infection status. In an embodiment, positive results do not rule out bacterial infection or co-infection with other viruses. Also, in certain embodiments, negative results may not preclude infection with at least one of SARS-CoV-2, Influenza A, Influenza B and/or RSV infection and should not be used as the sole basis for patient management decisions. In an embodiment, negative results are combined with clinical observations, patient history, and epidemiological information.

In certain embodiments, the method utilizes at least one of the following controls:
Internal Control—RNase P (RP) control in clinical samples. The RP primer and probe set is included in each run to test for human RP, which controls for specimen quality and demonstrates that nucleic acid was generated by the extraction process. Or another internal control may be used.
Positive Template Control—contains in vitro transcribed template (e.g., SARS-CoV-2 Influenza A, Influenza B and/or RSV) RNA with genomic regions targeted by the method. The positive control may be used to monitor for failures of RT-PCR reagents and reaction conditions.
Negative Extraction Control (NEC)—In an embodiment, this may be a previously characterized negative patient sample. Used as an extraction control and positive control for the RP primer and probe set.
No Template (Negative) Control—Nuclease-free, molecular-grade water used to monitor non-specific amplification, cross-contamination during experimental setup, and nucleic acid contamination of reagents.

### Compositions to Detect Respiratory Virus Infection

Also disclosed herein are compositions for performing any of the disclosed methods or running any of the disclosed systems. In some embodiments, the compositions are formulated as kits. In an embodiment, the compositions and/or kits comprise reagents for detecting the presence or absence of a respiratory virus in a sample from a subject. The composition and/or kit may comprise reagents or components for obtaining a sample from the subject, such as nasal swabs, buffer solutions, storage solutions and the like. In certain embodiments, the reagents or components for obtaining a sample from the subject are formulated for self-collection of the sample. The composition and/or kit may further comprise reagents and/or components for treating the sample with heat to inactivate any pathogen (including the viruses being assayed) present in the sample, and/or reagents or components for treating the sample with a protease, and/or reagents or components for partially purifying the viruses to be assayed as discussed in detail herein. The composition and/or kit may further comprise reagents or components for extracting nucleic acid from the sample.

The composition and/or kit may further comprise reagents or components for generating viral-specific cDNA and/or performing RT-PCR. Thus, the composition and/or kit may further comprise reagents or components for quantitative RT-PCR amplification, using primers specific to nucleic acid sequences specific for at least one, or two, or all three of SARS-CoV-2, Influenza A, Influenza B and/or RSV RNA, and in some embodiments, an internal probe (see e.g., Table 1). In embodiment, the RT-PCR is one-step RT-PCR. The components for RT-PCR may be formulated such that the multiplex RT-PCR may be performed. In certain embodiments, the internal probe may be labeled with a reporter and a quencher dye such that amplification allows the 5′→3′ exonuclease activity of Taq polymerase release of the reporter dye to be monitored. Any reporter and quencher dyes in the art may be used. In certain embodiments, the reporter dye is FAM, YakYel, TexRed, or Cy5. Additionally, and/or alternatively, the quencher dye may be the double quenchers ZEN™ in combination with an Iowa Black quencher (e.g., IABKFQ), and/or TAO™ in combination with an Iowa Black quencher (e.g., IABKFQSp) (see e.g., Table 1). In certain embodiments, the composition and/or kit comprises reagents for amplification of a control gene that is present in the subject, but not the virus. For example, the control gene may be the human RNase P (RP) gene or another gene such as a housekeeping gene.

Thus, in certain embodiments, the primers for RT-PCR amplification of the Influenza A M1 gene sequences comprise SEQ ID NOs: 1, 2, 3 or 4 and/or the probe used for detection of amplification of the Influenza A M1 gene sequences comprises SEQ ID NO: 5. Additionally and/or alternatively, the primers for RT-PCR amplification of the Influenza B NS2 gene sequences may comprise SEQ ID NOs: 6 and 7 and/or the probe used for detection of amplification of the Influenza B NS2 gene sequences may comprise SEQ ID NO: 8. Additionally and/or alternatively, the primers for RT-PCR amplification of the RSV M gene sequences may comprise SEQ ID NOs: 9 and 10 and/or the probe used for detection of amplification of the RSV M gene sequences may comprise SEQ ID NO: 11. Additionally and/or alternatively, the primers for RT-PCR amplification of the SARS-CoV-2 N gene sequences may comprise SEQ ID NOs: 12 and 13 and/or the probe used for detection of amplification of SARS-CoV-2 N gene sequences may comprise SEQ ID NO: 14. Additionally and/or alternatively, the primers for RT-PCR amplification of RNase P gene sequences may comprise SEQ ID NOs: 15 and 16 and/or the probe used for detection of amplification of RNase P gene sequences may comprises SEQ ID NO: 17.

In an embodiment, the composition and/or kit is formulated for multiplex real-time RT-PCR. Thus, in certain embodiments, the composition and/or kit is formulated such that cDNA (or RNA for one-step RT-PCR) from a single sample may be combined with primers and probes for at least two, or at least three, or all four of SARS-CoV-2, influenza A (Flu A), influenza B (Flu B), or Respiratory Syncytial Virus (RSV). The multiplex PCR may, in certain embodiments, comprise two different primers for Influenza A (Table 1) or any of the other viruses, and/or an internal control, such as RNase P (Table 1).

The composition and/or kit may further comprise at least one of: an Internal Control (e.g., RNase P); a Positive Template Control such as an in vitro transcribed template RNA, e.g., SARS-CoV-2, Influenza A, Influenza B and/or RSV that includes genomic regions targeted by the RT-PCR primers and probes; a Negative Extraction Control (NEC), e.g., a previously characterized negative sample; and/or a No Template (Negative) Control, e.g., nuclease-free, molecular-grade water.

The reagents and/or components of the composition and/or kit may be individually packaged. Also, the kit may comprise instructions for use.

### Systems to Detect Respiratory Virus Infection

Also disclosed are systems to perform any of the steps of any of the methods disclosed herein or to use any of the compositions and/or kits disclosed herein. Thus, disclosed is a system to determine whether a sample from a subject contains an infections respiratory virus comprising: a station and/or component for isolating RNA from a sample from the subject; a station and/or component for performing reverse transcriptase polymerase chain reaction (RT-PCR) amplification using primers and probes specific for at least two distinct respiratory viruses; and a station and/or component for determining the amount and/or absence and/or presence of at least one of the at least two distinct respiratory viruses in the sample.

In an embodiment, the primers and probes used for RT-PCR comprise at least one of SEQ ID NOs: 1-17. Thus, in certain embodiments, the primers for RT-PCR amplification of the Influenza A M1 gene sequences comprise SEQ ID NOs: 1, 2, 3 or 4 and/or the probe used for detection of amplification of the Influenza A M1 gene sequences comprises SEQ ID NO: 5. Additionally and/or alternatively, the primers for RT-PCR amplification of the Influenza B NS2 gene sequences may comprise SEQ ID NOs: 6 and 7 and/or the probe used for detection of amplification of the Influenza B NS2 gene sequences may comprise SEQ ID NO: 8. Additionally and/or alternatively, the primers for RT-PCR amplification of the RSV M gene sequences may comprise SEQ ID NOs: 9 and 10 and/or the probe used for detection of amplification of the RSV M gene sequences may comprise SEQ ID NO: 11. Additionally and/or alternatively, the primers for RT-PCR amplification of the SARS-CoV-2 N gene sequences may comprise SEQ ID NOs: 12 and 13 and/or the probe used for detection of amplification of SARS-CoV-2 N gene sequences may comprise SEQ ID NO: 14. Additionally and/or alternatively, the primers for RT-PCR amplification of RNase P gene sequences may comprise SEQ ID NOs: 15 and 16 and/or the probe used for detection of amplification of RNase P gene sequences may comprises SEQ ID NO: 17.

For example, disclosed is a system comprising a station and/or component to obtain and/or process a sample from a subject. In certain embodiments, the sample is an anterior nasal or nasopharyngeal swab. Or other sample types as disclosed herein may be used. The system may further comprise a component and/or station for generating virus-specific cDNA and/or performing RT-PCR using primers specific for at least two distinct respiratory viruses and/or a station and/or component for determining the amount of the at least two distinct respiratory viruses in the sample. For example, the system may comprise a station and/or component for isolating RNA from the sample. Also, the system may comprise a station and/or component for generating cDNA from the RNA and performing PCR amplification using viral-specific primers and probes. In an embodiment, the at least two distinct respiratory viruses comprise at least two of SARS-CoV-2, influenza A (Flu A), influenza B (Flu B), or Respiratory Syncytial Virus (RSV). Or other viruses may be detected. In certain embodiments, the system comprises a real-time RT-PCR assay. In an embodiment, the RT-PCR is one-step RT-PCR. Additionally and/or alternatively, the system may comprise a multiplex RT-PCR assay. Thus, in certain embodiments, cDNA (or RNA for one-step RT-PCR) from a single sample may be combined with primers and probes for at least two, or at least three, or all four of SARS-CoV-2, influenza A (Flu A), influenza B (Flu B), or Respiratory Syncytial Virus (RSV). The multiplex PCR may, in certain embodiments, comprise two different primers for Influenza A (Table 1) or any of the other viruses, and/or an internal control, such as RNase P (Table 1). Or other viruses may be detected.

In certain embodiments, the sample may be self-collected by the subject. Thus, in certain embodiments, samples may be individual anterior nasal swab specimens that are self-collected by individuals using a Labcorp COVID-19+Flu+RSV Test Home Collection Kit (Rx) and/or a Pixel by Labcorp COVID-19+Flu+RSV Test Home Collection Kit (DTC). In certain embodiments, a Pixel by Labcorp COVID-19+Flu+RSV Test Home Collection Kit is available direct-to-consumer through a Pixel website. Individuals (i.e., the subject) may be asked screening questions using a questionnaire on the Pixel website when ordering the kit. A lab order may be generated by the Physician Wellness Network (PWN) and the individual may perform the sample collection and mail it back to the testing lab.

In certain embodiments, the primers used for RT-PCR amplify SARS-CoV-2 nucleocapsid (N) gene nucleic acid. Additionally, and/or alternatively, the primers used for RT-PCR amplify influenza A virus matrix 1 (M1) gene nucleic acid. Additionally, and/or alternatively the primers used for RT-PCR amplify influenza B virus nonstructural 2 (NS2) gene nucleic acid. Additionally, and/or alternatively, the primers used for RT-PCR amplify Respiratory Syncytial Virus matrix (M) gene nucleic acid. Or other targets for each of these viruses may be used.

In certain embodiments, the system comprises a component for amplification of a control gene that is present in the subject, but not the pathogen. The internal control may be used to determine whether any of the steps of sample isolation, RNA isolation, RT-PCR and or data analysis have been performed according to predetermined standards. For example, the control gene may be the human RNase P (RP) gene or another human gene such as a housekeeping gene involved in basic cell maintenance. Or other control genes may be used. In certain embodiments, the primers and probes shown in Table 1 are used. Or other primers and probes may be used.

In certain embodiments, the system detects Influenza A virus (Flu A), Influenza B virus (Flu B), and Respiratory Syncytial Virus (RSV), in addition to SARS-CoV-2. In certain embodiments, the disclosed Seasonal Respiratory Virus RT-PCR Test is a multiplex test intended for the simultaneous qualitative detection and differentiation of RNA from SARS-CoV-2, Flu A, Flu B, and RSV from anterior nasal or NP swabs. Clinical signs and symptoms of respiratory infections caused by any of these viruses can be very similar, but treatment and quarantine requirements can be quite different. Thus, the ability to quickly differentiate between them can be critically important. The disclosed systems have the added benefit of use with anterior nasal swab specimens that are either self-collected by individuals at home or by their healthcare provider.

Also, the system may comprise a computer for running any of the stations and/or components of the system and/or analyzing the results. In certain embodiments, the station and/or component for analyzing the results may comprise a computer-program product tangibly embodied in a non-transitory machine-readable storage medium, including instructions configured to run any of the stations/components of the system and/or perform a step or steps of the methods.

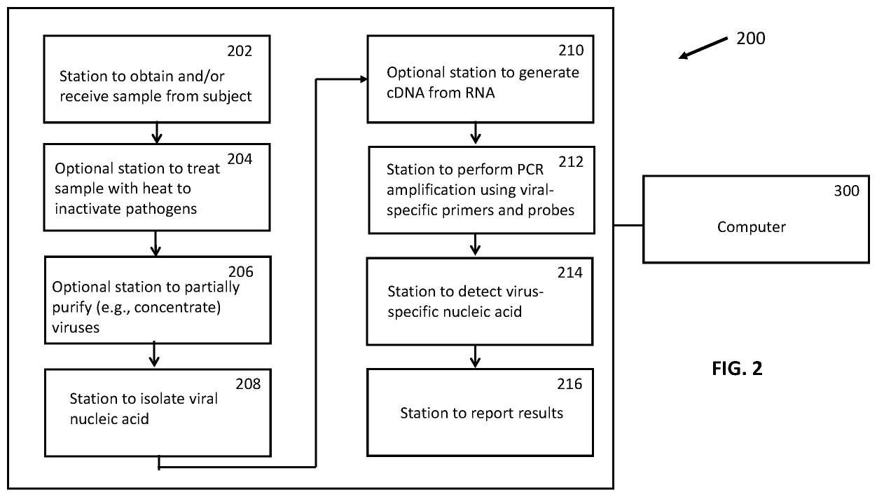

FIG. 2 illustrates one embodiment of a system (200) for detection and identification of seasonal virus infection. The illustration in FIG. 2 is not limited to a specific order of the components and/or stations. Thus, the system may comprise a station and/or component for obtaining and/or receiving a sample from a subject (202). For example, in many cases samples may be obtained from the subject (e.g., by a medical professional, caregiver or the subject themselves) at a site remote from the testing area and sent to the testing area. The sample may be an anterior nasal swab, or a nasopharyngeal or oropharyngeal swab, sputum, a lower respiratory tract aspirate, a bronchoalveolar lavage, or a nasopharyngeal wash/aspirate or nasal aspirate. In certain embodiments, the sample is an anterior nasal or nasopharyngeal swab. Or other types of samples may be used.

The sample may be self-collected by the subject. Thus, in certain embodiments, samples may be individual anterior nasal swab specimens that are self-collected by individuals using a Labcorp COVID-19+Flu+RSV Test Home Collection Kit (Rx) and/or a Pixel by Labcorp COVID-19+Flu+RSV Test Home Collection Kit (DTC) as disclosed herein.

The system may, in certain embodiments, have a station and/or component for treating the sample to inactivate pathogens present in the sample (204). In alternate embodiments, the sample is heated to at least 60 degrees C., or at least 65 degrees C., or at least 70 degrees C., or at least 75 degrees C. for a designated time. The sample may be heated for at least 10 minutes, or at least 20 minutes, or at least 30 minutes, or at least 40 minutes, or at least 50 minutes, or for 1 hour or more. In an embodiment, the sample may be heated at 65 degrees C. for about 30 minutes. In certain embodiments, the system may have a station for adding a protease, e.g., proteinase K or another protease. This station (not shown in FIG. 2) may be prior to, after, or part of the station for heat-inactivation.

Also optionally, the system may have a station and/or component to partially purify (e.g., concentrate) the viruses to be assayed (206). This station may be prior to, after, or part of the station for heat-inactivation. For example, for isolation of viral particles, the samples may be subjected to concentration (e.g., purification) of the virus using a matrix designed to bind viral particles (e.g., Nanotrap® Virus Capture Kit, Ceres Nanosciences, Inc.). Or other methods of purification may be used. Using such a matrix, elution of viral RNA from the concentrated viral particles may be performed a temperature of 90-99 degrees C. for at least 3 minutes. In an embodiment, elution may be performed at 95 degrees C. for about 5 minutes.

The system may also have a station and/or component for isolating nucleic acid (e.g., RNA) from the sample (208). For example, in certain embodiments, the station for isolating nucleic acid may comprise a semi-automated sample extraction protocol as for example, a ThermoFisher Scientific KingFisher Flex Magnetic Particle processor.

Where the nucleic acid is RNA, the system may have a station and/or component for generating cDNA from the RNA (210). The system may also have a station and/or component for performing amplification using viral-specific primers and probes to detect the at least one respiratory virus (212). In an embodiment, the RT-PCR is one-step RT-PCR such that the station and/or component to generate cDNA is the same as the station to amplify the viral-specific (i.e., SARS-Cov-2, Influenza A, Influenza B and/or RSV) nucleic acid sequences. In an embodiment, the RT-PCR is real-time RT-PCR. For example, in one embodiment, quantitative (i.e., real-time) PCR amplification, using primers specific to nucleic acid sequences in the pathogen and an internal probe may be used. In certain embodiments, RT-PCR plates are set-up using a Hamilton Microlab STAR liquid handling system and amplification/detection is performed with an Applied Biosystems QuantStudio7 Flex (software version 1.3). In an embodiment, the internal probe may be labeled with a reporter and a quencher dye such that amplification allows the 5′→3′ exonuclease activity of Taq polymerase to release the reporter dye, thereby allowing amplification to be monitored. Any reporter and quencher dyes in the art may be used. In certain embodiments, the reporter dye is FAM, YakYel, TexRed, or Cy5. Additionally, and/or alternatively, the quencher dye may be the double quenchers ZEN™ in combination with an Iowa Black quencher (e.g., IABKFQ), and/or TAO™ in combination with an Iowa Black quencher (e.g., IABKFQSp) (see e.g., Table 1). In certain embodiments, detecting further comprises amplification of a control gene that is present in the subject, but not the virus. For example, the control gene may be the human RNase P (RP) gene or another gene such as a housekeeping gene.

In an embodiment, the system station and/or component for RT-PCR comprises multiplex RT-PCR (e.g., multiplex real-time RT-PCR). Thus, in certain embodiments, cDNA (or RNA for one-step RT-PCR) from a single sample may be combined with primers and probes for at least two, or at least three, or all four of SARS-CoV-2, influenza A (Flu A), influenza B (Flu B), or Respiratory Syncytial Virus (RSV). The multiplex PCR may, in certain embodiments, comprise two different primers for Influenza A (Table 1) or any of the other viruses, and/or an internal control, such as RNase P (see e.g., Table 1 for primers and probes).

The system may further comprise as reagents, at least one of: an Internal Control (e.g., RNase P); a Positive Template Control (e.g., in vitro transcribed template for at least one of SARS-CoV-2, Influenza A, Influenza B and/or RSV RNA with genomic regions targeted by the method); a Negative Extraction Control (NEC) (e.g., a previously characterized negative sample); and/or a No Template (Negative) Control (e.g., nuclease-free, molecular-grade water).

Also, the system may have a station for determining the presence and/or absence and/or amount of viral-specific nucleic acid in the sample (214) as for example, by analysis of the results of the multiplex PCR. Such analysis may comprise review of the data to determine the level of detection for at least one of SARS-CoV-2, Influenza A, Influenza B and/or RSV. In an embodiment, the level of detection is measured by a cycle threshold (Ct) value. In certain embodiments, assay targets will be reported as detected (+) if they produce a Ct value of ≤40 or not detected (−) if they produce a Ct value of >40. Such analysis may comprise determining whether expected results are obtained for the internal control (e.g., human RNase P), a positive template control, a negative extraction control, and/or a no template control. In certain embodiments, tests that are negative for all targets are only valid if the RNase P internal control has a Ct value of ≤40. In certain embodiments, any test resulting as invalid is repeated at least once. Examples of potential results and corresponding result interpretation are shown in Table 5. In certain embodiments, the limit of detection (LoD) for each of SARS-CoV-2, Influenza A, Influenza B and/or RSV is <10 copies per microliter (μL). Example LoD results are shown in Tables 8 and 9.

The system may also include a station to report results to the subject, or his or her health care provider, or other medical professional (216). Results may be delivered to the subject electronically. In an embodiment, a PWN will follow up all positive and indeterminate or invalid test results by contacting the subject.

The disclosed systems can detect a plurality of respiratory virus isolates and/or strains. For example, for Influenza A, the method may detect A/California/07/09 (H1N1), A/Canada/6294/09 (H1N1), A/HongKong/4801/14 (H3N2), A/Mexico/4108/09 (H1N1), A/Michigan/45/15 (H1N1), A/Perth/16/09 (H3N2), A/Perth/16/09 (H3N2), A/Singapore/63/04 (H1N1), A/Switzerland/9715293/13 (H3N2), A/Texas/50/12 (H3N2), and/or A/Wisconsin/67/05 (H3N2). For influenza B, the method may detect B/Alabama/2/17 (Victoria), B/Brisbane/60/2008 (Victoria), B/Florida/78/2015 (Victoria), B/Washington/02/2019 (Victoria), B/Wisconsin/1/2010 (Yamagata), and/or B/Utah/9/2014 (Yamagata). For RSV, the method may detect RSV A 3/2015 Isolate #3, RSV A 2014 Isolate 341, RSV A 2013 Isolate, RSV A 2006 Isolate, RSV B CH93(18)-18, RSV B 3/2015 Isolate #1, and/or RSV B WV/14617/85. For SARS-CoV-2, the method may detect SARS-CoV-2 (Hong Kong/VM20001061/2020), SARS-CoV-2 (Isolate: Italy-INMI1), SARS-CoV-2 Variant B.1.351 SA, and/or SARS-CoV-2 Variant B.1.1.7 ENG. See e.g., Table 12. Or other strains and/or isolates may be detected.

The disclosure contemplates that certain of the stations as illustrated may be combined as a single station and/or component. For example, and not to be limiting, the stations for RNA isolation, cDNA preparation and RT-PCR (e.g., one-step RT-PCR) may be combined as a single station and/or component. Or the stations and/or components for heat-inactivation and partial purification of the viruses may be combined. Also, as illustrated in FIG. 2, any of the stations and/or components may be automated, robotically controlled, and/or controlled at least in part by a computer (300) and/or programmable software. Thus, the system may comprise a computer-program product tangibly embodied in a non-transitory machine-readable storage medium, including instructions configured to run the system or any part of the system and/or perform a step or steps of the methods or to use any of the kits or compositions of any of the disclosed embodiments. In some embodiments, a system is provided that includes one or more data processors and a non-transitory computer readable storage medium containing instructions which, when executed on the one or more data processors, cause the one or more data processors to perform part or all of one or more method steps or processes disclosed herein.

For example, disclosed is a system comprising one or more data processors, and a non-transitory computer readable storage medium containing instructions which, when executed on the one or more data processors, cause the one or more data processors to perform actions to direct at least one of the steps of obtaining a sample from the subject; isolating RNA from the sample; performing RT-PCR using primers and probes specific for at least two of SARS-CoV-2, Influenza A, Influenza B and/or RSV, and determining the presence or absence or amount of the at least two of SARS-CoV-2, Influenza A, Influenza B and/or RSV nucleic acid in the sample. In certain embodiments, the RT-PCR is real-time (quantitative) RT-PCR. Additionally, and/or alternatively, the RT-PCR may be multiplex RT-PCR.

Also disclosed is a computer-program product tangibly embodied in a non-transitory machine-readable storage medium, including instructions configured to run the systems and/or perform a step or steps of the methods of any of the disclosed embodiments. For example, in certain embodiments, the computer-program product tangibly embodied in a non-transitory machine-readable storage medium, includes instructions configured to cause one or more data processors to perform actions to direct at least one of the steps of obtaining a sample from the subject; isolating RNA from the sample; performing RT-PCR using primers and probes specific for at least two of SARS-CoV-2, Influenza A, Influenza B and/or RSV, and determining the presence and/or absence and/or amount of the at least two SARS-CoV-2, Influenza A, Influenza B and/or RSV nucleic acid in the sample. In certain embodiments, the RT-PCR is real-time (quantitative) RT-PCR. Additionally, and/or alternatively, the RT-PCR may be multiplex RT-PCR.

The systems and computer products may perform any of the methods disclosed herein. One or more embodiments described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

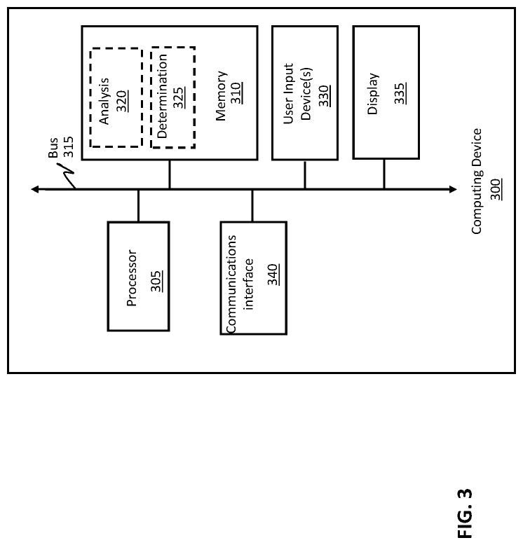

FIG. 3 shows a block diagram of an analysis system (300) used for detection and/or quantification of a pathogen. As illustrated in FIG. 3, modules, engines, or components (e.g., program, code, or instructions) executable by one or more processors may be used to implement the various subsystems of an analyzer system according to various embodiments. The modules, engines, or components may be stored on a non-transitory computer medium. As needed, one or more of the modules, engines, or components may be loaded into system memory (e.g., RAM) and executed by one or more processors of the analyzer system. In the example depicted in FIG. 3, modules, engines, or components are shown for implementing the methods of the disclosure.

Thus, FIG. 3 illustrates an example computing device (300) suitable for use with systems and the methods according to this disclosure. The example computing device (300) includes a processor (305) which is in communication with the memory (310) and other components of the computing device (300) using one or more communications buses (315). The processor (305) is configured to execute processor-executable instructions stored in the memory (310) to perform one or more methods or operate one or more stations for detecting pathogen levels according to different examples, such as those in FIGS. 1-2 or disclosed elsewhere herein. In this example, the memory (310) may store processor-executable instructions (325) that can analyze (320) RT-PCR results for at least two of SARS-CoV-2, Influenza A, Influenza B and/or RSV, as discussed herein.

The computing device 300 in this example may also include one or more user input devices (330), such as a keyboard, mouse, touchscreen, microphone, or the like, to accept user input. The computing device (300) may also include a display (335) to provide visual output to a user such as a user interface. The computing device (300) may also include a communications interface (340). In some examples, the communications interface (340) may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### EXAMPLES

### Example 1—Test Principle

The test kit is dispensed to patients when prescribed by a physician (e.g., using a test provider interface to order diagnostic tests). The kit is intended for use by individuals to self-collect nasal swab specimens at home, when determined by a healthcare provider to meet CDC clinical criteria and CDC epidemiological criteria to aid in the diagnosis of SARS-CoV-2, Influenza A, Influenza B and/or RSV infection. Once the physician order is placed, the home collection kit can be mailed to the patient, to perform the sample collection and mail it back to the test agency. Results are reported back to the ordering physician and to the patient via an automated patient portal.

The kit is composed of a shipping box, pre-labeled return envelope, directions, specimen collection materials (nasal swab and saline tube), a specimen biohazard bag and confirmation form/registration card. Instructions are included in the kit to direct the home users on how to appropriately collect the nasal swab specimen and place it in the saline transport tube, how to properly package the specimen and how to mail the specimen back to the laboratory.

The Seasonal Respiratory Virus RT-PCR Test is a multiplex real-time reverse transcription polymerase chain reaction (rRT-PCR) test. The test primer and probe sets are designed to detect the nucleocapsid gene of SARS-CoV-2, matrix 1 gene of Influenza A, nonstructural 2 gene of Influenza B, and matrix gene of RSV in anterior nasal samples collected from individuals suspected of infection by their healthcare provider. This test is for patients who meet CDC clinical criteria (e.g., exposure, signs and symptoms associated with SARS-CoV-2, Influenza A/B, or RSV infection) and/or CDC epidemiological criteria (e.g., history of residence in or travel to a geographic region with active SARS-CoV-2, Influenza A/B, or RSV transmission at the time of travel, or other epidemiologic criteria for which with SARS-CoV2, Influenza A/B, or RSV testing may be indicated) to aid in the diagnosis of with SARS-CoV2, Influenza A/B, or RSV infection.

### Example 2—Test Steps

Nucleic acids were isolated and purified from 200 uL of anterior nasal swab sample using a ThermoFisher MagMax Viral Pathogen Extraction Kit (Catalog #: A48310, A42352, A48383) and a ThermoFisher Scientific KingFisher Flex Magnetic Particle processor. Samples were eluted in a 50 uL volume. RT-PCR plates were set-up using the Hamilton Microlab STAR liquid handling system by combining 5 uL of the purified, extracted nucleic acids with 15 uL of RT-PCR Master mix. See Table 2 for the RT-PCR master mix components.

TABLE 2
RT-PCR Master Mix Recipe

| μL per reaction | Reagent |
|---|---|
| 8.00 | Sterile, distilled water |
| 5.00 | ThermoFisher TaqPath 1-Step Multiplex |
| | Master Mix 4x - No ROX |
| | (Catalog # A28523) |
| 2.00 | Primer/Probe Mix (IDT) |
| 15.00 | TOTAL VOLUME |

Samples were placed on the Applied Biosystems QuantStudio7 Flex (QS7) instrument for reverse transcription, amplification and detection. The QS7 cycling conditions are listed in Table 3.

TABLE 3
RT-PCR Cycling Conditions

| Step | Cycles | ° C. | Hold | | Ramp Rate |
|---|---|---|---|---|---|
| Reverse Transcription | 1 | 48 | 15 | min | 100% |
| Denaturation | 1 | 95 | 2 | min | 100% |
| Amplification | 40 | 95 | 1 | s | 100% |
| | | 55 | 20 | s | 100% |

During RT-PCR, the probe anneals to a specific target sequence located between the forward and reverse primers. During the extension phase of the PCR cycle, the 5′ nuclease activity of Taq polymerase degrades the probe, causing the reporter dye to separate from the quencher dye, generating a fluorescent signal. With each cycle, additional reporter dye molecules are cleaved from their respective probes, increasing the fluorescence intensity. Fluorescence intensity was monitored at each PCR cycle by the Applied Biosystems QuantStudio7 Flex (QS7) instrument.

### Example 3—Control Materials

Controls run with each extracted set of samples (e.g., 93 samples) were as follows:

No Template Control (NTC): A negative control consisting of molecular grade nuclease free water was used to detect contamination during extraction and RT-PCR. The NTC should test negative for all targets in the assay, including the RNase P internal control.

Negative Extraction Control (NEC): A previously characterized negative patient sample served as a negative control to monitor for any cross-contamination that may occur during the testing procedure.

Positive Control: A positive template control was used to verify functionality of PCR reagents and that the assay run was performing as intended. The positive control consisted of DNA gBlocks (synthesized double-stranded DNA) purchased from Integrated DNA Technologies. Each target sequence (Influenza A, Influenza B, RSV and SARS-CoV-2) was included on a separate gBlock and contained viral genomic sequences encompassing the primer/probe binding regions. The four control gBlocks were added together to form a multiplex control. 500 copies of the Influenza A, Influenza B, and SARS-CoV-2 gBlock and 5,000 copies of the RSV gBlock were added to each positive control reaction. This equates to approximately 16× the LoD for Influenza A, Influenza B, and SARS-CoV-2 and 40× the LoD for RSV. The positive control should test positive for all 4 viral targets.

Internal control (IC): An additional primer/probe set with a distinct fluorophore (Cy5) was included in the primer/probe mix that targets human RNase P, which is present in human specimens. This IC was used for every sample processed and to verify that nucleic acid of adequate quality and quantity was present. This also served as the extraction and reverse transcription control to ensure that samples resulting as negative for test targets contained nucleic acid for testing and that viral RNA was successfully transcribed into DNA.

### Example 4—Interpretation of Results

The control results in Table 4 were required for each run in order for the run to be deemed valid. If the plate controls were not valid, the plate was repeated.

TABLE 4
Required Control Results

| | Influenza A | Influenza B | RSV | SARS-CoV-2 | RNaseP |
|---|---|---|---|---|---|
| | Ct | Ct | Ct | Ct | Ct |
| Control | Value | Value | Value | Value | Value |
| NTC | >40* | >40 | >40 | >40 | >40 |
| NEC | >40 | >40 | >40 | >40 | ≤40 |
| Positive | ≤40 | ≤40 | ≤40 | ≤40 | >40 |
| Control | | | | | |

*an “undetermined” result is the same as “no signal” or a Ct > 40

If a test run was valid, the results for individual patient samples could be interpreted and reported. Assay targets were reported as detected (+) if they produced a Ct value of ≤40 or not detected (−) if they produced a Ct value of >40. Tests negative for all targets were only deemed valid if the RNase P internal control Ct value was ≤40. Any test resulting as invalid was repeated once. Potential results and corresponding result interpretation are listed in Table 5.

TABLE 5
Seasonal Respiratory Virus RT-PCR Test Interpretation

| | | | | | Result | |
|---|---|---|---|---|---|---|
| Flu | Flu | | | | Inter- | |
| A | B | RSV | SC2 | RP | pretation | Action |
| + | − | − | − | + or − | Influenza A | Report results to sender and |
| | | | | | detected | appropriate public health |
| | | | | | | authorities |
| − | + | − | − | + or − | Influenza B | Report results to sender and |
| | | | | | detected | appropriate public health |
| | | | | | | authorities |
| − | − | + | − | + or − | RSV | Report results to sender and |
| | | | | | detected | appropriate public health |
| | | | | | | authorities |
| − | − | − | + | + or − | SARS- | Report results to sender and |
| | | | | | COV-2 | appropriate public health |
| | | | | | detected | authorities |
| + | + | − | − | + or − | Influenza | Report results to sender and |
| | | | | | A and | appropriate public health |
| | | | | | Influenza B | authorities |
| | | | | | detected | |
| + | − | + | − | + or − | Influenza | Report results to sender and |
| | | | | | A and | appropriate public health |
| | | | | | RSV | authorities |
| | | | | | detected | |
| + | − | − | + | + or − | Influenza | Report results to sender and |
| | | | | | A and | appropriate public health |
| | | | | | SARS- | authorities |
| | | | | | COV-2 | |
| | | | | | detected | |
| − | + | + | − | + or − | Influenza | Report results to sender and |
| | | | | | B and | appropriate public health |
| | | | | | RSV | authorities |
| | | | | | detected | |
| − | + | − | + | + or − | Influenza | Report results to sender and |
| | | | | | B and | appropriate public health |
| | | | | | SARS- | authorities |
| | | | | | COV-2 | |
| | | | | | detected | |
| − | − | + | + | + or − | RSV and | Report results to sender and |
| | | | | | SARS- | appropriate public health |
| | | | | | CoV-2 | authorities |
| | | | | | detected | |
| + | + | + | − | + or − | Influenza A, | Report results to sender and |
| | | | | | Influenza B, | appropriate public health |
| | | | | | and | authorities |
| | | | | | RSV | |
| | | | | | detected | |
| + | − | + | + | + or − | Influenza A, | Report results to sender and |
| | | | | | RSV | appropriate public health |
| | | | | | and SARS- | authorities |
| | | | | | COV-2 | |
| | | | | | detected | |
| − | + | + | + | + or − | Influenza B, | Report results to sender and |
| | | | | | RSV | appropriate public health |
| | | | | | and SARS- | authorities |
| | | | | | COV-2 | |
| | | | | | detected | |
| + | + | − | + | + or − | Influenza A, | Report results to sender and |
| | | | | | Influenza B, | appropriate public health |
| | | | | | and | authorities |
| | | | | | SARS- | |
| | | | | | COV-2 | |
| | | | | | detected | |
| + | + | + | + | + or − | Influenza A, | Report results to sender and |
| | | | | | Influenza B, | appropriate public health |
| | | | | | RSV, | authorities |
| | | | | | and SARS- | |
| | | | | | COV-2 | |
| | | | | | detected | |
| − | − | − | − | + | Negative | Report results to sender. |
| − | − | − | − | − | Invalid | Repeat extraction and RT- |
| | | | | | | PCR. If the repeated result |
| | | | | | | remains invalid, consider |
| | | | | | | collecting a new specimen |
| | | | | | | from the patient. |

### Example 5—Performance Evaluation

Limit of Detection

The analytical sensitivities of the Seasonal Respiratory Virus RT-PCR Test were determined utilizing the virus strains listed in Table 6 for Limit of Detection (LoD). Table 7 lists the associated stock concentrations in copies/μL and in TCID₅₀/μL. These same strain lots were also used in subsequent sections of the examples.

TABLE 6
Strains Used in LoD Determination

| Viral Strain | Source | Catalog # | Lot # | Format |
|---|---|---|---|---|
| A/Nebraska/14/2018 (H1N1) | Microbiologics | N/A* | D2013A | Culture Fluid |
| A/Hong Kong/2671/2019 (H3N2) | Virapur | N/A | C2030D | Culture Fluid |
| B/Colorado/06/2017 (Victoria) | Virapur | N/A | B1904S1 | Culture Fluid |
| B/Phu ket/3073/2013 (Yamagata) | Virapur | N/A | B1904N | Culture Fluid |
| RSV A2 | Virapur | N/A | K1907B | Culture Fluid |
| RSV B/Washington | Virapur | N/A | E1831C | Culture Fluid |
| SARS-CoV-2 USA-WA1/2020 | BEI Resources | NR-52350 | 70033928 | Heat-Inact. |
| | | | | Virus |

*not applicable

TABLE 7
Viral Stock Concentrations

Stock Concentration

| Viral Strain | TCID₅₀/μL | copies/μL |
|---|---|---|
| A/Nebraska/14/2018 (H1N1) | 6.3 × 10⁴ | 4.60 × 10⁶ |
| A/Hong Kong/2671/2019 (H3N2) | 6.3 × 10³ | 2.27 × 10⁶ |
| B/Colorado/06/2017 (Victoria) | 1.4 × 10⁵ | 1.13 × 10⁶ |
| B/Phu ket/3073/2013 (Yamagata) | 1.4 × 10⁵ | 7.23 × 10⁶ |
| RSV A2 | 6.3 × 10³ | 4.99 × 10⁶ |
| RSV B Washington | 4.6 × 10³ | 4.98 × 10⁶ |
| SARS-CoV-2 USA-WA1/2020* | 18 | 3.40 × 10⁵ |

*titer determined prior to inactivation

Virus stocks were diluted in 2-fold increments over the expected LoD concentration ranges using pooled negative anterior nasal swab samples collected in saline. A total of 20 samples of each virus at each dilution were tested. The results of the LoD study are summarized in Table 8. Table 9 summarizes the analytical sensitivities for each virus strain tested.

TABLE 8
Limit of Detection Results

| | Concentration | | Posi- | % | |
|---|---|---|---|---|---|
| | copies/ | TCID₅₀/ | tive/ | Detec- | Mean |
| Viral Strain | μL | μL | Total | tion | Ct* |
| A/Nebraska/14/2018 | 12.50 | 0.1712 | 20/20 | 100.0 | 32.33 |
| (H1N1) | 6.25 | 0.0856 | 20/20 | 100.0 | 33.23 |
| | 3.13 | 0.0428 | 20/20 | 100.0 | 34.53 |
| | 1.56 | 0.0214 | 20/20 | 100.0 | 35.59 |
| | 0.78 | 0.0107 | 17/20 | 85.0 | 37.88 |
| A/Hong Kong/2671/2019 | 12.50 | 0.0347 | 20/20 | 100.0 | 31.90 |
| (H3N2) | 6.25 | 0.0173 | 20/20 | 100.0 | 32.81 |
| | 3.13 | 0.0087 | 20/20 | 100.0 | 33.65 |
| | 1.56 | 0.0043 | 20/20 | 100.0 | 35.16 |
| | 0.78 | 0.0022 | 20/20 | 100.0 | 36.88 |
| | 0.39 | 0.0011 | 3/20 | 15.0 | 39.08 |
| | 0.19 | 0.0005 | 0/20 | 0.0 | N/A** |
| B/Colorado/06/2017 | 3.13 | 0.3878 | 20/20 | 100.0 | 37.70 |
| (Victoria) | 1.56 | 0.1939 | 19/20 | 95.0 | 38.27 |
| | 0.78 | 0.0969 | 16/20 | 80.0 | 38.90 |
| | 0.39 | 0.0485 | 7/20 | 35.0 | 39.43 |
| | 0.19 | 0.0242 | 1/20 | 5.0 | N/A |
| B/Phu ket/3073/2013 | 12.50 | 0.2420 | 20/20 | 100.0 | 31.22 |
| (Yamagata) | 6.25 | 0.1210 | 20/20 | 100.0 | 32.10 |
| | 3.13 | 0.0605 | 20/20 | 100.0 | 33.35 |
| | 1.56 | 0.0303 | 20/20 | 100.0 | 34.34 |
| | 0.78 | 0.0151 | 20/20 | 100.0 | 35.71 |
| | 0.39 | 0.0076 | 7/20 | 35.0 | 38.78 |
| | 0.19 | 0.0038 | 3/20 | 15.0 | 39.91 |
| RSV A2 | 12.50 | 0.0158 | 20/20 | 100.0 | 32.55 |
| | 6.25 | 0.0079 | 20/20 | 100.0 | 33.65 |
| | 3.13 | 0.0039 | 20/20 | 100.0 | 34.57 |
| | 1.56 | 0.0020 | 20/20 | 100.0 | 36.21 |
| | 0.78 | 0.0010 | 18/20 | 90.0 | 37.49 |
| RSV B Washington | 12.50 | 0.0115 | 20/20 | 100.0 | 35.06 |
| | 6.25 | 0.0058 | 20/20 | 100.0 | 36.03 |
| | 3.13 | 0.0029 | 18/20 | 90.0 | 37.31 |
| | 1.56 | 0.0014 | 13/20 | 65.0 | 38.30 |
| | 0.78 | 0.0007 | 5/20 | 25.0 | 38.46 |
| SARS-CoV-2 | 12.50 | 0.00066 | 20/20 | 100.0 | 33.00 |
| USA-WA1/2020 | 6.25 | 0.00033 | 20/20 | 100.0 | 33.73 |
| | 3.13 | 0.00017 | 20/20 | 100.0 | 34.92 |
| | 1.56 | 0.00008 | 20/20 | 100.0 | 36.10 |
| | 0.78 | 0.00004 | 18/20 | 90.0 | 37.92 |

*calculated using only replicates returning a positive result
**not applicable

TABLE 9
Summary of Analytical Sensitivities

Concentration

| Virus Strain | copies/μL | TCID₅₀/μL |
|---|---|---|
| A/Nebraska/14/2018 (H1N1) | 1.56 | 0.0214 |
| A/Hong Kong/2671/2019 (H3N2) | 0.78 | 0.0022 |
| B/Colorado/06/2017 (Victoria) | 1.56 | 0.1939 |
| B/Phu ket/3073/2013 (Yamagata) | 0.78 | 0.0151 |
| RSV A2 | 1.56 | 0.0020 |
| RSV B Washington | 6.25 | 0.0058 |
| SARS-CoV-2 USA-WA1/2020 | 1.56 | 0.00008 |

Co-Formulated Limit of Detection

A co-formulated LoD study was performed for the Seasonal Respiratory Virus RT-PCR Test. The Influenza A, Influenza B, and RSV strains with the LoDs showing the least sensitivity were combined with SARS-CoV-2 USA-WA1/2020 for testing. Strains were diluted to their respective LoD using pooled negative anterior nasal swab samples collected in saline and 20 replicates tested. The results of the co-formulated testing are summarized in Table 10. The assay LoDs for Flu A, Flu B, and SARS-CoV-2 were not affected by the multiplex testing. However, RSV demonstrated a drop in sensitivity.

TABLE 10
LoD Results for Flu A, Flu B, RSV, and
SARS-CoV-2 Strain Co-formulation

| | | Positives/Total | | | |
|---|---|---|---|---|---|
| Strain Combination | copies/μL | Flu A | Flu B | RSV | SARS-CoV-2 |
| A/Nebraska/14/2018 | 1.56 | 19/20 | 20/20 | 2/20 | 20/20 |
| B/Colorado/06/2017 | 1.56 | | | | |
| RSV B Washington | 6.25 | | | | |
| SARS-CoV-2 USA- | 1.56 | | | | |
| WA1/2020 | | | | | |

The study was repeated removing the RSV strain (RSV B Washington) from the combination. The assay LoDs for Flu A, Flu B, and SARS-CoV-2 were not affected when the three targets were co-formulated. The results are summarized in Table 11.

TABLE 11
LoD Results for Flu A, Flu B and SARS-COV-2 Strain Co-formulation

| | | | | | | SARS- | |
|---|---|---|---|---|---|---|---|
| | | Flu A | | Flu B | | COV-2 | |
| | | Pos/ | Ave. | Pos/ | Ave. | Pos/ | Ave. |
| Strain Combination | copies/μL | total | Ct | total | Ct | total | Ct |
| A/Nebraska/14/2018 | 1.56 | 20/20 | 35.94 | 20/20 | 34.60 | 20/20 | 35.78 |
| B/Colorado/06/2017 | 1.56 | | | | | | |
| SARS-COV-2 USA- | 1.56 | | | | | | |
| WA1/2020 | | | | | | | |

Inclusivity (Analytical Sensitivity)

Inclusivity of the Seasonal Respiratory Virus RT-PCR Test was demonstrated by testing multiple strains of Influenza A (H1N1 and H3N2), Influenza B (Yamagata and Victoria lineages), RSV (A and B) and SARS-CoV-2. Virus stocks were individually diluted to 3× the LoD (Flu A, Flu B, and SARS-CoV-2 each at 4.58 cp/μL and RSV at 18.75 cp/μL) using pooled negative nasal swab samples and tested in triplicate. All strains were detected at 3×LoD. The results of the inclusivity testing are summarized in Table 12. The corresponding tested concentrations in TCID₅₀/L are also listed in the table.

TABLE 12
Summary of Inclusivity Results

| | | Positive Replicates/Total | | | |
|---|---|---|---|---|---|
| | | | | | SARS- |
| Viral Strain | TCID₅₀/μL | Flu A | Flu B | RSV | CoV-2 |
| A/California/07/09 (H1N1) | 0.305 | 3/3 | 0/3 | 0/3 | 0/3 |
| A/Canada/6294/09 (H1N1) | 0.227 | 3/3 | 0/3 | 0/3 | 0/3 |
| A/HongKong/4801/14 (H3N2) | 0.203 | 3/3 | 0/3 | 0/3 | 0/3 |
| A/Mexico/4108/09 (H1N1) | 0.021 | 3/3 | 0/3 | 0/3 | 0/3 |
| A/Michigan/45/15 (H1N1) | 0.009 | 3/3 | 0/3 | 0/3 | 0/3 |
| A/Perth/16/09 (H3N2) | 0.071 | 3/3 | 0/3 | 0/3 | 0/3 |
| A/Singapore/63/04 (H1N1) | 0.010 | 3/3 | 0/3 | 0/3 | 0/3 |
| A/Switzerland/9715293/13 (H3N2) | 0.071 | 3/3 | 0/3 | 0/3 | 0/3 |
| A/Texas/50/12 (H3N2) | 0.210 | 3/3 | 0/3 | 0/3 | 0/3 |
| A/Wisconsin/67/05 (H3N2) | 0.491 | 3/3 | 0/3 | 0/3 | 0/3 |
| B/Alabama/2/17 (Victoria) | 0.006 | 0/3 | 3/3 | 0/3 | 0/3 |
| B/Brisbane/60/2008 (Victoria) | 0.006 | 0/3 | 3/3 | 0/3 | 0/3 |
| B/Florida/78/2015 (Victoria) | 0.238 | 0/3 | 3/3 | 0/3 | 0/3 |
| B/Washington/02/2019 (Victoria) | 22.003 | 0/3 | 3/3 | 0/3 | 0/3 |
| B/Wisconsin/1/2010 (Yamagata) | 0.169 | 0/3 | 3/3 | 0/3 | 0/3 |
| B/Utah/9/2014 (Yamagata) | 0.001 | 0/3 | 3/3 | 0/3 | 0/3 |
| RSV A 3/2015 Isolate #3 | 0.002 | 0/3 | 0/3 | 3/3 | 0/3 |
| RSV A 2014 Isolate 341 | 0.005 | 0/3 | 0/3 | 3/3 | 0/3 |
| RSV A 2013 Isolate | 0.0013 | 0/3 | 0/3 | 3/3 | 0/3 |
| RSV A 2006 Isolate | 0.0003 | 0/3 | 0/3 | 3/3 | 0/3 |
| RSV B CH93(18)-18 | 0.0037 | 0/3 | 0/3 | 3/3 | 0/3 |
| RSV B 3/2015 Isolate #1 | 0.0026 | 0/3 | 0/3 | 3/3 | 0/3 |
| RSV B WV/14617/85 | 0.0054 | 0/3 | 0/3 | 3/3 | 0/3 |
| SARS-CoV-2 (Hong | 0.006 | 0/3 | 0/3 | 0/3 | 3/3 |
| Kong/VM20001061/2020) | | | | | |
| SARS-CoV-2 (Isolate: Italy-INMI1) | 0.102 | 0/3 | 0/3 | 0/3 | 3/3 |
| SARS-CoV-2 Variant B.1.351 SA | 0.0005 | 0/3 | 0/3 | 0/3 | 3/3 |
| SARS-CoV-2 Variant B.1.1.7 ENG | 0.0004 | 0/3 | 0/3 | 0/3 | 3/3 |

Cross-Reactivity (Analytical Specificity)

Cross-reactivity of the Seasonal Respiratory Virus RT-PCR Test was evaluated by testing a panel of 4 organisms consisting of 22 virus, 19 bacteria, and two fungus strains representing common respiratory pathogens or flora commonly present in respiratory tract. All organisms were intact except for human coronavirus HKU1. Viruses were tested at concentrations of ≥1×10⁵ copies/mL or TCID₅₀/mL, except where otherwise noted. Bacteria and fungus strains were tested at a concentration of 1×10⁶ copies/mL, cfu/mL or IFU/mL, except where noted. Organisms were diluted to the listed concentrations using pooled negative anterior nasal swab samples (saline) and tested in triplicate. No cross-reactivity with common non-target respiratory organisms was detected and target viruses only yielded a positive test result for the appropriate virus. The results are summarized in Table 13.

TABLE 13
Cross-reactivity with Common Respiratory Organisms

| | | | Positive Replicates/Total | | | |
|---|---|---|---|---|---|---|
| | | | | | | SARS- |
| Organism | Concentration | | Flu A | Flu B | RSV | CoV-2 |
| Influenza A Virus | 1 × 10⁶ | cp/mL | 3/3 | 0/3 | 0/3 | 0/3 |
| Influenza B Virus | 1 × 10⁶ | cp/mL | 0/3 | 3/3 | 0/3 | 0/3 |
| Respiratory Syncytial Virus A | 1 × 10⁶ | cp/mL | 0/3 | 0/3 | 3/3 | 0/3 |
| Respiratory Syncytial Virus B | 1 × 10⁶ | cp/mL | 0/3 | 0/3 | 3/3 | 0/3 |
| SARS-CoV-2 | 1 × 10⁶ | cp/mL | 0/3 | 0/3 | 0/3 | 3/3 |
| SARS Coronavirus | 1 × 10⁴ TCID₅₀/mL | | 0/3 | 0/3 | 0/3 | 0/3 |
| Bordetella pertussis | 1 × 10⁶ | cfu/mL | 0/3 | 0/3 | 0/3 | 0/3 |
| Candida albicans | 1 × 10⁶ | cfu/mL | 0/3 | 0/3 | 0/3 | 0/3 |
| Chlamydophila pneumoniae | 1 × 10⁶ | IFU/mL | 0/3 | 0/3 | 0/3 | 0/3 |
| Corynebacterium diphtheriae | 1 × 10⁶ | cfu/mL | 0/3 | 0/3 | 0/3 | 0/3 |
| Escherichia coli | 1 × 10⁶ | cfu/mL | 0/3 | 0/3 | 0/3 | 0/3 |
| Haemophilus influenzae | 1 × 10⁶ | cfu/mL | 0/3 | 0/3 | 0/3 | 0/3 |
| Lactobacillus acidophilus | 1 × 10⁶ | cfu/mL | 0/3 | 0/3 | 0/3 | 0/3 |
| Legionella longbeachae | 1 × 10⁶ | cfu/mL | 0/3 | 0/3 | 0/3 | 0/3 |
| Legionella pneumophila | 1 × 10⁶ | cfu/mL | 0/3 | 0/3 | 0/3 | 0/3 |
| Moraxella catarrhalis | 1 × 10⁶ | cfu/mL | 0/3 | 0/3 | 0/3 | 0/3 |
| Mycoplasma pneumoniae | 1 × 10⁶ | cfu/mL | 0/3 | 0/3 | 0/3 | 0/3 |
| Neisseria lactamica | 1 × 10⁶ | cfu/mL | 0/3 | 0/3 | 0/3 | 0/3 |
| Neisseria meningitidis | 1 × 10⁶ | cfu/mL | 0/3 | 0/3 | 0/3 | 0/3 |
| Pneumocystis carinii | 1 × 10⁶ | cp/mL | 0/3 | 0/3 | 0/3 | 0/3 |
| Pseudomonas aeruginosa | 1 × 10⁶ | cfu/mL | 0/3 | 0/3 | 0/3 | 0/3 |
| Staphylococcus epidermidis | 1 × 10⁶ | cfu/mL | 0/3 | 0/3 | 0/3 | 0/3 |
| Streptococcus pneumoniae | 1 × 10⁶ | cfu/mL | 0/3 | 0/3 | 0/3 | 0/3 |
| Streptococcus salivarius | 1 × 10⁶ | cfu/mL | 0/3 | 0/3 | 0/3 | 0/3 |
| Human Coronavirus 229E | 1 × 10⁵ TCID₅₀/mL | | 0/3 | 0/3 | 0/3 | 0/3 |
| Adenovirus Type 1 | 1 × 10⁵ TCID₅₀/mL | | 0/3 | 0/3 | 0/3 | 0/3 |
| Adenovirus Type 7 | 1 × 10⁵ TCID₅₀/mL | | 0/3 | 0/3 | 0/3 | 0/3 |
| Cytomegalovirus | 1 × 10⁵ | cp/mL | 0/3 | 0/3 | 0/3 | 0/3 |
| Epstein Barr Virus | 1 × 10⁵ | cp/mL | 0/3 | 0/3 | 0/3 | 0/3 |
| Enterovirus D68 | 1 × 10⁵ TCID₅₀/mL | | 0/3 | 0/3 | 0/3 | 0/3 |
| Human coronavirus HKU1* | 1 × 10⁴ | cp/mL | 0/3 | 0/3 | 0/3 | 0/3 |
| Human Metapneumovirus | 1 × 10⁵ TCID₅₀/mL | | 0/3 | 0/3 | 0/3 | 0/3 |
| Mycobacterium tuberculosis | >5 × 10³ | cfu/mL | 0/3 | 0/3 | 0/3 | 0/3 |
| MERS-coronavirus | 1 × 10⁵ TCID₅₀/mL | | 0/3 | 0/3 | 0/3 | 0/3 |
| Human Coronavirus NL63 | 1 × 10⁴ TCID₅₀/mL | | 0/3 | 0/3 | 0/3 | 0/3 |
| Human Coronavirus OC43 | 1 × 10⁴ TCID₅₀/mL | | 0/3 | 0/3 | 0/3 | 0/3 |
| Human Parainfluenza Virus 1 | 1 × 10⁵ TCID₅₀/mL | | 0/3 | 0/3 | 0/3 | 0/3 |
| Human Parainfluenza Virus 2 | 1 × 10⁵ TCID₅₀/mL | | 0/3 | 0/3 | 0/3 | 0/3 |
| Human Parainfluenza Virus 3 | 1 × 10⁵ TCID₅₀/mL | | 0/3 | 0/3 | 0/3 | 0/3 |
| Human Parainfluenza Virus 4 | 1 × 10⁴ TCID₅₀/mL | | 0/3 | 0/3 | 0/3 | 0/3 |
| Human rhinovirus 61 | 1 × 10⁵ TCID₅₀/mL | | 0/3 | 0/3 | 0/3 | 0/3 |
| Staphylococcus aureus | 1 × 10⁶ | cfu/mL | 0/3 | 0/3 | 0/3 | 0/3 |
| Streptococcus pyogenes | 1 × 10⁶ | cfu/mL | 0/3 | 0/3 | 0/3 | 0/3 |

*isolated RNA - synthetic genomic construct

Microbial Interference

Assay target viruses were tested in the presence of the organisms listed in Table 14. All potentially interfering organisms were intact except for human coronavirus HKU1. Testing was performed by co-spiking viruses A/Nebraska/14/2018, B/Colorado/06/2017, and SARS-CoV-2 USA-WA1/2020 (at 3× the LOD—4.68 cp/μL each) into samples containing each potentially interfering organism. RSV B Washington was tested individually at 3× the LOD (18.75 cp/μL) in the presence of each potentially interfering organism. Non-target viruses were tested at concentrations of 1×10⁵ copies/mL or TCID₅₀/mL, except where otherwise noted. Bacteria and fungus strains were tested at concentrations of 1×10⁶ copies/mL, cfu/mL or IFU/mL, except where noted. Target viruses and potentially interfering organisms were diluted using pooled negative anterior nasal swab samples (saline) and tested in triplicate. All target viruses were detected at 3×LOD in the presence of the other listed organisms. The results are summarized in Table 14.

TABLE 14
Interference with Other Common Respiratory Organisms

| | | | Positive Replicates/Total | | | |
|---|---|---|---|---|---|---|
| | | | | | | SARS- |
| Organism | Concentration | | Flu A | Flu B | RSV | CoV-2 |
| None | N/A | | 3/3 | 3/3 | 3/3 | 3/3 |
| SARS Coronavirus | 1 × 10⁴ TCID₅₀/mL | | 3/3 | 3/3 | 3/3 | 3/3 |
| Bordetella pertussis | 1 × 10⁶ | cfu/mL | 3/3 | 3/3 | 3/3 | 3/3 |
| Candida albicans | 1 × 10⁶ | cfu/mL | 3/3 | 3/3 | 3/3 | 3/3 |
| Chlamydophila pneumoniae | 1 × 10⁶ | IFU/mL | 3/3 | 3/3 | 3/3 | 3/3 |
| Corynebacterium diphtheriae | 1 × 10⁶ | cfu/mL | 3/3 | 3/3 | 3/3 | 3/3 |
| Escherichia coli | 1 × 10⁶ | cfu/mL | 3/3 | 3/3 | 3/3 | 3/3 |
| Haemophilus influenzae | 1 × 10⁶ | cfu/mL | 3/3 | 3/3 | 3/3 | 3/3 |
| Lactobacillus acidophilus | 1 × 10⁶ | cfu/mL | 3/3 | 3/3 | 3/3 | 3/3 |
| Legionella longbeachae | 1 × 10⁶ | cfu/mL | 3/3 | 3/3 | 3/3 | 3/3 |
| Legionella pneumophila | 1 × 10⁶ | cfu/mL | 3/3 | 3/3 | 3/3 | 3/3 |
| Moraxella catarrhalis | 1 × 10⁶ | cfu/mL | 3/3 | 3/3 | 3/3 | 3/3 |
| Mycoplasma pneumoniae | 1 × 10⁶ | cfu/mL | 3/3 | 3/3 | 3/3 | 3/3 |
| Neisseria lactamica | 1 × 10⁶ | cfu/mL | 3/3 | 3/3 | 3/3 | 3/3 |
| Neisseria meningitidis | 1 × 10⁶ | cfu/mL | 3/3 | 3/3 | 3/3 | 3/3 |
| Pneumocystis carinii | 1 × 10⁶ | nuclei/mL | 3/3 | 3/3 | 3/3 | 3/3 |
| Pseudomonas aeruginosa | 1 × 10⁶ | cfu/mL | 3/3 | 3/3 | 3/3 | 3/3 |
| Staphylococcus epidermidis | 1 × 10⁶ | cfu/mL | 3/3 | 3/3 | 3/3 | 3/3 |
| Streptococcus pneumoniae | 1 × 10⁶ | cfu/mL | 3/3 | 3/3 | 3/3 | 3/3 |
| Streptococcus salivarius | 1 × 10⁶ | cfu/mL | 3/3 | 3/3 | 3/3 | 3/3 |
| Human Coronavirus 229E | 1 × 10⁵ TCID₅₀/mL | | 3/3 | 3/3 | 3/3 | 3/3 |
| Adenovirus Type 1 | 1 × 10⁵ TCID₅₀/mL | | 3/3 | 3/3 | 3/3 | 3/3 |
| Adenovirus Type 7 | 1 × 10⁵ TCID₅₀/mL | | 3/3 | 3/3 | 3/3 | 3/3 |
| Cytomegalovirus | 1 × 10⁵ | cp/mL | 3/3 | 3/3 | 3/3 | 3/3 |
| Epstein Barr Virus | 1 × 10⁵ | cp/mL | 3/3 | 3/3 | 3/3 | 3/3 |
| Enterovirus D68 | 1 × 10⁵ TCID₅₀/mL | | 3/3 | 3/3 | 3/3 | 3/3 |
| Human coronavirus HKU1* | 1 × 10⁴ | cp/mL | 3/3 | 3/3 | 3/3 | 3/3 |
| Human Metapneumovirus | 1 × 10⁵ TCID₅₀/mL | | 3/3 | 3/3 | 3/3 | 3/3 |
| Mycobacterium tuberculosis | >5 × 10³ | cfu/mL | 3/3 | 3/3 | 3/3 | 3/3 |
| MERS-coronavirus | 1 × 10⁵ TCID₅₀/mL | | 3/3 | 3/3 | 3/3 | 3/3 |
| Human Coronavirus NL63 | 1 × 10⁴ TCID₅₀/mL | | 3/3 | 3/3 | 3/3 | 3/3 |
| Human Coronavirus OC43 | 1 × 10⁴ TCID₅₀/mL | | 3/3 | 3/3 | 3/3 | 3/3 |
| Human Parainfluenza Virus 1 | 1 × 10⁵ TCID₅₀/mL | | 3/3 | 3/3 | 3/3 | 3/3 |
| Human Parainfluenza Virus 2 | 1 × 10⁵ TCID₅₀/mL | | 3/3 | 3/3 | 3/3 | 3/3 |
| Human Parainfluenza Virus 3 | 1 × 10⁵ TCID₅₀/mL | | 3/3 | 3/3 | 3/3 | 3/3 |
| Human Parainfluenza Virus 4 | 1 × 10⁴ TCID₅₀/mL | | 3/3 | 3/3 | 3/3 | 3/3 |
| Human rhinovirus 61 | 1 × 10⁵ TCID₅₀/mL | | 3/3 | 3/3 | 3/3 | 3/3 |
| Staphylococcus aureus | 1 × 10⁶ | cfu/mL | 3/3 | 3/3 | 3/3 | 3/3 |
| Streptococcus pyogenes | 1 × 10⁶ | cfu/mL | 3/3 | 3/3 | 3/3 | 3/3 |

*isolated RNA - synthetic genomic construct

Co-Infection (Competitive Interference)

To evaluate coinfection inhibition, two target strains were combined for testing such that one was at a low concentration (3×L/D), and one was at a high concentration (10⁶ copies/mL). Samples were tested in triplicate. The strains utilized are listed in Table 15 and were diluted in pooled negative anterior nasal swab samples (saline). Inhibition was observed only for Flu A, RSV A, and RSV B at 3×LoD in the presence of high levels of Flu B (Table 15).

TABLE 15
Co-infection with an Interfering Target at 10⁶ copies/mL

| | Interfering Targets (10⁶ copies/mL) | | | | | |
|---|---|---|---|---|---|---|
| | None | Flu A | Flu B | RSV A | RSV B | Sars-COV-2 |
| Target | Positive Replicates/Total | | | | | |
| A/Nebraska/14/ | 3/3 | N/A* | 1/3 | 3/3 | 3/3 | 3/3 |
| 2018 3X LOD | | | | | | |
| B/Colorado/06/ | 3 /3 | 3/3 | N/A | 3/3 | 3/3 | 3/3 |
| 2017 3X LOD | | | | | | |
| RSV A2 3X LOD | 3/3 | 3/3 | 0/3 | N/A | N/A | 3/3 |
| RSV B Washington | 3/3 | 3/3 | 1/3 | N/A | N/A | 3/3 |
| 3X LOD | | | | | | |
| SARS-COV-2 USA- | 3/3 | 3/3 | 3 /3 | 3/3 | 3/3 | N/A |
| WA1/2020 3X LOD | | | | | | |
| None | 0/3 | 3/3 | 3/3 | 3/3 | 3/3 | 3/3 |

*Not applicable

Testing was performed again with Flu A, RSV A, and RSV B at 3×LoD in the presence of Flu B at 105 copies/mL (Table 16). No inhibition was observed at this concentration of Flu B.

TABLE 16
Co-infection with an Interfering Target at 10⁵ copies/mL

| | | Positive Replicates/ |
|---|---|---|
| Low Target | Interfering Target | Total |
| A/Nebraska/14/2018 3X LOD | Flu B 10⁵ | 3/3 |
| RSV A2 3X LOD | copies/mL | 3/3 |
| RSV B Washington 3X LOD | | 3/3 |

Interfering Substances (Endogenous and Exogenous)

Endogenous and exogenous substances that may be present in nasal swab specimens were evaluated for potential interference in the Seasonal Respiratory Virus RT-PCR Test. Testing was performed by co-spiking A/Nebraska/14/2018, B/Colorado/06/2017, and SARS-CoV-2 USA-WA1/2020 (at 3× the LoD—4.68 cp/μL each) into samples separately containing the substances listed in Table 17. RSV B Washington was spiked individually at 3× the LoD (18.75 cp/μL) in the presence of each substance. Target viruses and potentially interfering substances were diluted to the listed concentrations using pooled negative anterior nasal swab samples (saline) and tested in triplicate. None of the substances inhibited the assay at the concentrations tested. The results are summarized in Table 17.

TABLE 17
Interference with Endogenous and Exogenous Substances

| | Description/ | | | Positive Replicates/Total | | | | |
|---|---|---|---|---|---|---|---|---|
| | Active | Conc. in | | | | | SARS- | |
| Substance | Ingredient | Sample | | Flu A | Flu B | RSV | CoV-2 | None |
| Neo-Synephrine | Phenylephrine | 15% | v/v | 3/3 | 3/3 | 3/3 | 3/3 | 0/3 |
| | hydrochloride 0.5% | | | | | | | |
| Affrin | Oxymetazoline | 5% | v/v | 3/3 | 3/3 | 3/3 | 3/3 | 0/3 |
| | hydrochloride | | | | | | | |
| | 0.05% | | | | | | | |
| Saline Nasal | NaCl (0.65%), | 15% | v/v | 3/3 | 3/3 | 3/3 | 3/3 | 0/3 |
| Spray | phenylcarbinol & | | | | | | | |
| | benzalkonium | | | | | | | |
| | chloride | | | | | | | |
| Zicam Nasal | Galphimia glauca, | 15% | v/v | 3/3 | 3/3 | 3/3 | 3/3 | 0/3 |
| Spray | Histaminum | | | | | | | |
| | hydrochloricum, | | | | | | | |
| | Luffa operculata, | | | | | | | |
| | Sulpher | | | | | | | |
| Flonase | Fluticasone | 2.5% | v/v | 3/3 | 3/3 | 3/3 | 3/3 | 0/3 |
| | propionate 50 mcg | | | | | | | |
| Rhinocort | Budesonide 32 mcg | 5% | v/v | 3/3 | 3/3 | 3/3 | 3/3 | 0/3 |
| Nasacort | Triamcinolone | 2.5% | v/v | 3/3 | 3/3 | 3/3 | 3/3 | 0/3 |
| | acetonide 55 mcg | | | | | | | |
| Nasal | Dexamethasone | 10 | mcg/mL | 3/3 | 3/3 | 3/3 | 3/3 | 0/3 |
| Corticosteroid | 0.1 mg/mL | | | | | | | |
| Nasal | Mometasone | 5% | v/v | 3/3 | 3/3 | 3/3 | 3/3 | 0/3 |
| Corticosteroid | furoate 50 mcg | | | | | | | |
| Qnasl | Beclomethasone | 5% | v/v | 3/3 | 3/3 | 3/3 | 3/3 | 0/3 |
| | dipropionate | | | | | | | |
| | 80 mcg | | | | | | | |
| Chloraseptic | Benzocaine 15 mg, | 1% | w/v | 3/3 | 3/3 | 3/3 | 3/3 | 0/3 |
| max | Menthol 10 mg | | | | | | | |
| Antibiotic nasal | Mupirocin 20 mg/g | 10 | mg/mL | 3/3 | 3/3 | 3/3 | 3/3 | 0/3 |
| ointment | | | | | | | | |
| Relenza | Zanamivir 5 mg | 5 | mg/mL | 3/3 | 3/3 | 3/3 | 3/3 | 0/3 |
| Antiviral Drug | Oseltamivir | 10 | mcg/mL | 3/3 | 3/3 | 3/3 | 3/3 | 0/3 |
| | phosphate 6 mg/mL | | | | | | | |
| Antibiotic, | Tobramycin | 2 | mg/mL | 3/3 | 3/3 | 3/3 | 3/3 | 0/3 |
| systemic | (40 mg) | | | | | | | |
| FluMist* | Live intranasal | N/A | | N/A | N/A | N/A | N/A | N/A |
| | influenza virus | | | | | | | |
| Ayr | Nasal Gel | 1% | w/v | 3/3 | 3/3 | 3/3 | 3/3 | 0/3 |
| NasoGEL | Nasal Gel | 0.5% | w/v | 3/3 | 3/3 | 3/3 | 3/3 | 0/3 |
| Mucin | Bovine Sub- | 0.1 | mg/mL | 3/3 | 3/3 | 3/3 | 3/3 | 0/3 |
| | maxillary Gland | | | | | | | |
| Blood | Human | 2% | v/v | 3/3 | 3/3 | 3/3 | 3/3 | 0/3 |

*FluMist was not available and so could not be tested

Shipping Sample Stability

To test the shipping stability of FluA, FluB, RSV, and SARS-CoV-2 when using either the Labcorp COVID-19+Flu+RSV Test Home Collection Kit or the Pixel by Labcorp COVID-19+Flu+RSV Test Home Collection Kit, stability studies mimicking worst-case scenario summer and winter conditions were performed. See Tables 18 and 19 for the conditions tested. The Labcorp COVID-19+Flu+RSV Test Home Collection Kit and the Pixel by Labcorp COVID-19+Flu+RSV Test Home Collection Kit have the same components and collection instructions; therefore, these sample stability studies are supportive of both collection kits.

TABLE 18
Summer Stability Study Profile

| Temperature | Cycle Period | Cycle Period (Hours) | Total Time (Hours) |
|---|---|---|---|
| 40° C. | 1 | 8 | 8 |
| 22° C. | 2 | 4 | 12 |
| 40° C. | 3 | 2 | 14 |
| 30° C. | 4 | 36 | 50 |
| 40° C. | 5 | 6 | 56 |

TABLE 19
Winter Stability Study Profile

| Temperature | | Cycle Period | Cycle Period (Hours) | Total Time (Hours) |
|---|---|---|---|---|
| −10° | C. | 1 | 8 | 8 |
| 18° | C. | 2 | 4 | 12 |
| −10° | C. | 3 | 2 | 14 |
| 10° | C. | 4 | 36 | 50 |
| −10° | C. | 5 | 6 | 56 |

A SARS-CoV-2-positive remnant patient sample, a live Influenza A culture (A/Nebraska/14/2018), a live Influenza B culture (B/Colorado/06/2017) and a live RSV B culture (RSV B Washington) were separately added to individual negative anterior nasal swab samples (saline) to yield concentrations at 2× the LoD (3.12, 3.12, 3.12 and 12.50 cp/μL, respectively) and 10× the LoD (15.6, 15.6, 15.6 and 62.5 cp/μL, respectively). For each viral type, twenty 2×LoD samples and ten 10×LoD samples were independently subjected to the summer and winter profiles. In addition, 10 negative samples were included in each excursion.

Samples were tested at 0-hour and after the 56-hour temperature excursion using the Seasonal Respiratory Virus RT-PCR Test. The mean Ct values at T=56 hour was compared to the mean Ct values at time 0. All samples remained positive after each excursion (Tables 20 and 21) except for one RSV replicate at 2×LoD during the summer excursion. The mean Ct values at T=56 hour for all targets were within 3.0 Ct compared to time 0, indicating acceptable specimen stability under the conditions tested. All negative samples were non-detectable for all targets and had passing RNase P internal control results.

Together, these data support the stability of anterior nasal swab samples collected with either the Labcorp COVID-19+Flu+RSV Test Home Collection Kit and the Pixel by Labcorp COVID-19+Flu+RSV Test Home Collection Kit for up to 56 hours in an ambient temperature shipping environment.

TABLE 20
Summer Excursion Results

| | T = 0 hr 2X LoD | | T = 56 hr 2X LoD | | T = 0 hr 10X LoD | | T = 56 hr 10X LoD | |
|---|---|---|---|---|---|---|---|---|
| | Pos/ | Mean | Pos/ | Mean | Pos/ | Mean | Pos/ | Mean |
| Virus | Total | Ct | Total | Ct | Total | Ct | Total | Ct |
| Flu A | 20/20 | 33.84 | 20/20 | 35.67 | 10/10 | 31.64 | 10/10 | 32.94 |
| Flu B | 20/20 | 32.91 | 20/20 | 35.43 | 10/10 | 31.18 | 10/10 | 33.13 |
| RSV | 20/20 | 34.63 | 19/20 | 36.09* | 10/10 | 31.57 | 10/10 | 33.23 |
| SARS-COV-2 | 20/20 | 31.46 | 20/20 | 31.01 | 10/10 | 29.21 | 10/10 | 29.37 |

*Negative result omitted from calculation

TABLE 21
Winter Excursion Results

| | T = 0 hr 2X LoD | | T = 56 hr 2X LoD | | T = 0 hr 10X LoD | | T = 56 hr 10X LoD | |
|---|---|---|---|---|---|---|---|---|
| | Pos/ | Mean | Pos/ | Mean | Pos/ | Mean | Pos/ | Mean |
| Virus | Total | Ct | Total | Ct | Total | Ct | Total | Ct |
| Flu A | 20/20 | 34.40 | 20/20 | 35.04 | 10/10 | 31.80 | 10/10 | 32.72 |
| Flu B | 20/20 | 33.29 | 20/20 | 35.01 | 10/10 | 31.29 | 10/10 | 32.46 |
| RSV | 20/20 | 33.31 | 20/20 | 35.10 | 10/10 | 30.67 | 10/10 | 31.96 |
| SARS-COV-2 | 20/20 | 30.69 | 20/20 | 31.25 | 10/10 | 28.15 | 10/10 | 28.27 |

Carry-Over/Cross Contamination

To demonstrate a lack of carry-over and cross-contamination for the Seasonal Respiratory Virus RT-PCR Test, alternating high positive samples and negative samples were assayed on six runs. Each run contained three replicates each of Flu A, Flu B, RSV A, RSV B, and SARS-CoV-2 at 1×10⁵ TCID₅₀/mL. Negatives consisted of pooled negative anterior nasal swab samples (saline). All negative samples were found negative for all viral targets, demonstrating no carry-over or cross-contamination during the assay procedure.

Precision

Within-lab precision of the Seasonal Respiratory Virus RT-PCR Test was assessed by repeat testing a panel of samples. Samples included co-spiked strains A/Nebraska/14/2018, B/Colorado/06/2017, and SARS-CoV-2 USA-WA1/2020 at 1×LoD (1.56 cp/μL each) and 3×LoD (4.68 cp/μL each) as well as RSV B Washington alone at 1×LoD (6.25 cp/μL) and 3×LoD (18.75 cp/μL). Dilutions were generated using pooled negative anterior nasal swab samples (saline). Negative replicate samples were included in the panel to monitor contamination.

Each of the 5 sample types was tested in quadruplicate for a total of 20 samples per run. Two operators performed separate runs each day over six non-consecutive days. In addition, 2 different instrument sets and 2 different reagent lots were used. See Table 22 for a summary of the testing parameters.

TABLE 22
Precision Testing Parameters

| | | | | | | Reps | Total |
|---|---|---|---|---|---|---|---|
| | | | | | | per | samples |
| Day | Run | Operator | Instrument | Lot | Samples | conc. | per run |
| 1 | 1 | 1 | 1 | 1 | 1. 1X LoD - | 4 | 20 |
| | 2 | 2 | 2 | 2 | FLUA/B/COVID | 4 | 20 |
| 2 | 1 | 1 | 1 | 2 | 2. 3X LoD - | 4 | 20 |
| | 2 | 2 | 2 | 1 | FLUA/B/COVID | 4 | 20 |
| 3 | 1 | 1 | 2 | 2 | 3. 1X LoD - RSV | 4 | 20 |
| | 2 | 2 | 1 | 1 | 4. 3X LoD - RSV | 4 | 20 |
| 4 | 1 | 1 | 2 | 1 | 5. Negative | 4 | 20 |
| | 2 | 2 | 1 | 2 | | 4 | 20 |
| 5 | 1 | 1 | 2 | 2 | | 4 | 20 |
| | 2 | 2 | 1 | 1 | | 4 | 20 |
| 6 | 1 | 1 | 2 | 1 | | 4 | 20 |
| | 2 | 2 | 1 | 2 | | 4 | 20 |

The positivity rate was calculated for each target at each concentration using the results generated from all days. In addition, the overall mean Ct, standard deviation (SD), and % coefficient of variation (%/CV) were calculated. All 3×LoD target replicates showed a 100% positivity rate and all 1×LoD target replicates showed a ≥95% positivity rate except for RSV which had a positivity rate of 91.7%. This is slightly below the expected rate of 95% and may have been due to slight variability introduced during sample dilution and processing. The negative samples achieved 100% negative results for all assay targets. See Table 23 for the summarized results.

TABLE 23
Summary of Within-Laboratory Precision

| | Concen- | Pos/ | Positivity Rate | Mean | | |
|---|---|---|---|---|---|---|
| Virus | tration | Total | (95% CI) | Ct | S.D. | % CV |
| Flu A | Negative | 0/48 | 0.0% (0.0-7.4) | N/A ** | N/A | N/A |
| | 1X LOD | 47/48 | 97.9% (89.1-99.6) | 37.55* | 1.10* | 2.94* |
| | 3X LOD | 48/48 | 100.0% (92.6-100.0) | 35.19 | 0.82 | 2.34 |
| Flu B | Negative | 0/48 | 0.0% (0.0-7.4) | N/A | N/A | N/A |
| | 1X LOD | 48/48 | 100.0% (92.6-100.0) | 35.89 | 0.71 | 1.97 |
| | 3X LOD | 48/48 | 100.0% (92.6-100.0) | 34.05 | 0.43 | 1.27 |
| RSV | Negative | 0/48 | 0.0% (0.0-7.4) | N/A | N/A | N/A |
| | 1X LOD | 44/48 | 91.7% (80.5-96.7) | 37.51* | 2.04* | 5.43* |
| | 3X LOD | 48/48 | 100.0% (92.6-100.0) | 33.81 | 2.12 | 6.27 |
| SARS- | Negative | 0/48 | 0.0% (0.0-7.4) | N/A | N/A | N/A |
| CoV-2 | 1X LOD | 48/48 | 100.0% (92.6-100.0) | 36.45 | 0.70 | 1.93 |
| | 3X LOD | 48/48 | 100.0% (92.6-100.0) | 34.76 | 0.64 | 1.84 |

*Ct values from replicates returning a negative result were not used in calculations
** Not applicable

Additionally, ANOVA analysis was performed to quantify between-day, between-operator, between-instrument, between-lot, and between-run precision. See Table 24 for a summary of the results.

TABLE 24
Summary of Precision Across Different Parameters

| | | Different | | Different | | Different | | Different | | Different | |
|---|---|---|---|---|---|---|---|---|---|---|---|
| | | Days | | Operators | | Instruments | | Reagent Lots | | Runs | |
| Virus | Conc. | SD | % CV | SD | % CV | SD | % CV | SD | % CV | SD | % CV |
| Flu A | 1X LOD | 0.90 | 2.40 | 0.67 | 1.77 | 1.33 | 3.55 | 1.40 | 3.73 | 0.93 | 2.46 |
| | 3X LOD | 0.97 | 2.75 | 1.89 | 5.38 | 2.20 | 6.25 | 0.12 | 0.34 | 0.84 | 2.40 |
| Flu B | 1X LOD | 1.32 | 3.68 | 0.05 | 0.14 | 0.03 | 0.10 | 0.10 | 0.28 | 0.65 | 1.82 |
| | 3X LOD | 0.76 | 2.22 | 0.11 | 0.31 | 0.25 | 0.74 | 0.95 | 2.79 | 0.72 | 2.11 |
| RSV | 1X LOD | 2.01 | 5.37 | 1.05 | 2.80 | 0.93 | 2.48 | 2.88 | 7.68 | 2.55 | 6.79 |
| | 3X LOD | 2.21 | 6.53 | 1.83 | 5.40 | 2.00 | 5.92 | 2.84 | 8.41 | 1.64 | 4.85 |
| SARS- | 1X LOD | 0.83 | 2.29 | 0.24 | 0.64 | 0.82 | 2.25 | 0.53 | 1.46 | 0.84 | 2.31 |
| CoV-2 | 3X LOD | 1.02 | 2.93 | 0.06 | 0.17 | 1.67 | 4.80 | 0.44 | 1.27 | 0.91 | 2.61 |

Fresh vs. Frozen Test Samples

A study was performed to determine the effect of sample freezing on detectability. Samples included 10 replicates of co-spiked strains A/Nebraska/14/2018, B/Colorado/06/2017, and SARS-CoV-2 USA-WA1/2020 at 1×LoD (1.56 cp/μL each) and 3×LoD (4.68 cp/μL each) as well as 10 replicates of RSV B Washington alone at 1×LoD (6.25 cp/μL) and at 3×LoD (18.75 cp/μL). Dilutions were generated using pooled negative anterior nasal swab samples (saline). In addition, 10 negative replicates were included to monitor contamination. Samples were tested immediately after generation and then after a freeze-thaw cycle. After freezing, all target replicates were still detectable and the mean Ct values for all targets were within 3.0 Ct, indicating minimal loss of sensitivity. All negatives returned negative target results. See Table 25 for a summary of the results.

TABLE 25
Fresh Versus Frozen Specimen Results

| | Pre-Freeze | | Post-Freeze | | Pre-Freeze | | Post-Freeze | |
|---|---|---|---|---|---|---|---|---|
| | 1X LoD | | 1X LoD | | 3X LOD | | 3X LOD | |
| | Pos/ | Mean | Pos/ | Mean | Pos/ | Mean | Pos/ | Mean |
| Virus | Total | Ct | Total | Ct | Total | Ct | Total | Ct |
| Flu A | 10/10 | 36.69 | 10/10 | 37.33 | 10/10 | 34.96 | 10/10 | 35.76 |
| Flu B | 10/10 | 35.33 | 10/10 | 37.24 | 10/10 | 33.89 | 10/10 | 35.40 |
| RSV | 10/10 | 34.44 | 10/10 | 36.94 | 10/10 | 33.30 | 10/10 | 35.44 |
| SARS-COV-2 | 10/10 | 36.85 | 10/10 | 35.89 | 10/10 | 35.12 | 10/10 | 34.15 |

### Example 6—Clinical Evaluation

Fifty FluA-positive, 30 FluB-positive, 30 RSV-positive, and 50 negative samples were tested using the Seasonal Respiratory Virus RT-PCR Test and also with the FDA-cleared Cobas® Liat Influenza A/B & RSV Test as a comparator. Clinical samples consisted of nasopharyngeal swabs collected in UTM that were all archived frozen in the previous 4 years. Collection dates are noted with the line data. Given the lack of low positive RSV samples, five unique RSV-positive samples were randomly selected and diluted in negative UTM to give low titer samples. The Cobas® Liat Test has mean Flu A, Flu B, and RSV Ct values of approximately 32.37, 31.70 and 32.87, respectively, at the corresponding LoDs. At least 20% of each of the target samples tested were low positives (within approximately ±3 Ct at the Liat LoDs). The positive predictive agreement (PPA) for Flu A, Flu B, and RSV were all 100%. The negative predictive agreement (NPA) was 100%. The lower bound 95% confidence interval for each target PPA was >88%. See Table 26 for the summarized results.

Ninety-three SARS-CoV-2-positive and 44 negative samples were tested with the Seasonal Respiratory Virus RT-PCR Test and also using the Hologic Panther Fusion® SARS-CoV-2 assay as a comparator. Clinical samples consisted of nasopharyngeal swabs collected in UTM that were archived frozen within the past 6 months. The Panther Fusion Assay has a SARS-CoV-2 Ct value of approximately 35.6 at the LoD. At least 20% of the SARS-CoV-2 samples were low positives (within a Panther Fusion® Ct value range of 32.6 and 38.6). The PPA for SARS-CoV-2 was found to be 96.7% with a lower bound 95% confidence interval of 90.9%. The NPA was 100%. Three of the negative samples had failing internal controls and were omitted from analysis. See Table 26 for the summarized results.

TABLE 26
Clinical Concordance Results for the Seasonal Respiratory Virus RT-PCR Test

| | | | | | SARS-COV-2 Samples | |
|---|---|---|---|---|---|---|
| | FLU A, Flu B and RSV Samples | | | | SARS- | |
| Target > | FluA | FluB | RSV | Negative | CoV-2 | Negative |
| Concordant | 50 | 30 | 30 | 50 | 90 | 41 |
| False Neg | 0 | 0 | 0 | 0 | 3 | 0 |
| False Pos | 0 | 0 | 0 | 0 | 0 | 0 |
| Failing RNaseP | 0 | 0 | 0 | 0 | 0 | 3 |
| Total | 50 | 30 | 30 | 50 | 93 | 44 |
| PPA | 100.0% | 100.0% | 100.0% | N/A | 96.7% | N/A |
| (95% CI) | (92.9-100) | (88.7-100) | (88.7-100) | | (90.9-98.9) | |
| NPA | NA | NA | NA | 100.0% | NA | 100.0% |
| (95% CI) | | | | (92.9-100) | | (91.4-100) |

### Example 7—Embodiments

As used below, any reference to methods or systems is understood as a reference to each of those methods or systems disjunctively (e.g., “Illustrative embodiment 1-4 is understood as illustrative embodiment 1, 2, 3, or 4.”).

Illustrative embodiment 1 is a method to determine whether a sample from a subject contains an infections respiratory virus comprising:
(a) obtaining the sample from a subject;
(b) isolating RNA from the sample;
(c) generating copy DNA (cDNA) from the RNA;
(d) performing PCR amplification of the cDNA using primers and probes specific for the at least two distinct respiratory viruses; and
(e) determining the amount and/or absence and/or presence of at least one of the at least two distinct respiratory viruses in the sample.

Illustrative embodiment 2 is the method of any preceding or subsequent illustrative method embodiment, wherein the at least two distinct respiratory viruses comprise at least two of SARS-CoV-2, influenza A (Flu A), influenza B (Flu B), or Respiratory Syncytial Virus (RSV).

Illustrative embodiment 3 is the method of any preceding or subsequent illustrative method embodiment, wherein the at least two distinct respiratory viruses comprise at least three of SARS-CoV-2, influenza A (Flu A), influenza B (Flu B), or Respiratory Syncytial Virus (RSV).

Illustrative embodiment 4 is the method of any preceding or subsequent illustrative method embodiment, wherein the at least two distinct respiratory viruses comprise each of SARS-CoV-2, influenza A (Flu A), influenza B (Flu B), or Respiratory Syncytial Virus (RSV).

Illustrative embodiment 5 is the method of any preceding or subsequent illustrative method embodiment, wherein the sample is an anterior nasal or nasopharyngeal (NP) swab.

Illustrative embodiment 6 is the method of any preceding or subsequent illustrative method embodiment, wherein the sample is self-collected by the subject.

Illustrative embodiment 7 is the method of any preceding or subsequent illustrative method embodiment, wherein the RT-PCR comprises a multiplex RT-PCR assay.

Illustrative embodiment 8 is the method of any preceding or subsequent illustrative method embodiment, wherein the RT-PCR is real-time RT-PCR.

Illustrative embodiment 9 is the method of any preceding or subsequent illustrative method embodiment, wherein the primers used for RT-PCR amplify SARS-CoV-2 nucleocapsid (N) gene nucleic acid.

Illustrative embodiment 10 is the method of any preceding or subsequent illustrative method embodiment, wherein the primers used for RT-PCR amplify influenza A virus matrix 1 (M1) gene nucleic acid.

Illustrative embodiment 11 is the method of any preceding or subsequent illustrative method embodiment, wherein the primers used for RT-PCR amplify influenza B virus nonstructural 2 (NS2) gene nucleic acid.

Illustrative embodiment 12 is the method of any preceding or subsequent illustrative method embodiment, wherein the primers used for RT-PCR amplify Respiratory Syncytial Virus matrix (M) gene nucleic acid.

Illustrative embodiment 13 is the method of any preceding or subsequent illustrative method embodiment, further comprising amplification of a control gene that is present in the subject, but not the pathogen.

Illustrative embodiment 14 is the method of any preceding or subsequent illustrative method embodiment, wherein the control gene is the human RNase P (RP) gene or another human gene such as a housekeeping gene involved in basic cell maintenance.

Illustrative embodiment 15 is the method of any preceding or subsequent illustrative method embodiment, wherein the primers for RT-PCR amplification of Influenza A M1 gene sequences comprise SEQ ID NOs: 1, 2, 3 or 4.

Illustrative embodiment 16 is the method of any preceding or subsequent illustrative method embodiment, wherein the probe used for detection of amplification of the Influenza A M1 gene sequences comprises SEQ ID NO: 5.

Illustrative embodiment 17 is the method of any preceding or subsequent illustrative method embodiment, wherein the primers for RT-PCR amplification of Influenza B NS2 gene sequences comprise SEQ ID NOs: 6 and 7.

Illustrative embodiment 18 is the method of any preceding or subsequent illustrative method embodiment, wherein the probe used for detection of amplification of the Influenza B NS2 gene sequences comprises SEQ ID NO: 8.

Illustrative embodiment 19 is the method of any preceding or subsequent illustrative method embodiment, wherein the primers for RT-PCR amplification of RSV M gene sequences comprise SEQ ID NOs: 9 and 10.

Illustrative embodiment 20 is the method of any preceding or subsequent illustrative method embodiment, wherein the probe used for detection of amplification of the RSV M gene sequences comprises SEQ ID NO: 11.

Illustrative embodiment 21 is the method of any preceding or subsequent illustrative method embodiment, wherein the primers for RT-PCR amplification of SARS-CoV-2 N gene sequences comprise SEQ ID NOs: 12 and 13.

Illustrative embodiment 22 is the method of any preceding or subsequent illustrative method embodiment, wherein the probe used for detection of amplification of the SARS-CoV-2 N gene sequences comprises SEQ ID NO: 14.

Illustrative embodiment 23 is the method of any preceding or subsequent illustrative method embodiment, wherein the primers for RT-PCR amplification of RNase P gene sequences comprise SEQ ID NOs: 15 and 16.

Illustrative embodiment 24 is the method of any preceding or subsequent illustrative method embodiment, wherein the probe used for detection of amplification of the RNase P gene sequences comprises SEQ ID NO: 17.

Illustrative embodiment 25 is the method of any preceding or subsequent illustrative method embodiment, wherein determining the amount or presence of at least one of the at least two distinct respiratory viruses in the sample comprises a positive result (+) if the at least one of the two distinct respiratory viruses produces a Ct of ≤40 and/or a negative or not detected result (−) if the at least one of the two distinct respiratory viruses produces a Ct of >40.

Illustrative embodiment 26 is the method of any preceding or subsequent illustrative method embodiment, wherein determining the amount or presence of at least one of the at least two distinct respiratory viruses in the sample comprises determining whether expected results are obtained for the internal control (e.g., human RNase P), and/or a positive template control (e.g., nucleic acid sequences specific to at least one of SARS-CoV-2, Influenza A, Influenza B and/or RSV), and/or a negative extraction control (e.g., a sample from an individual not infected with any of the viruses being tested for), and/or a no template control (e.g., water or buffer).

Illustrative embodiment 27 is the method of any preceding or subsequent illustrative method embodiment, wherein results that are negative for all targets are interpreted to be valid if the RNase P internal control produces a Ct≤40.

Illustrative embodiment 28 is the method of any preceding or subsequent illustrative method embodiment, wherein the limit of detection (LoD) for each of SARS-CoV-2, Influenza A, Influenza B and/or RSV is <10 copies per microliter (μL).

Illustrative embodiment 29 is the method of any preceding or subsequent illustrative method embodiment, wherein a positive predictive agreement (PPA) for Flu A, Flu B, and RSV is 100%, and/or a negative predictive agreement (NPA) for Flu A, Flu B, and RSV is 100%, and/or a PPA for SARS-CoV-2 is 96.7% with a lower bound 95% confidence interval of 90.9%, and/or a NPA for SARS-CoV-2 is 100%.

Illustrative embodiment 30 is the method of any preceding or subsequent illustrative method embodiment, wherein a reporter dye used for RT-PCR is FAM, YakYel, TexRed, or Cy5 and/or a quencher dye used for RT-PCR is the double quenchers ZEN™ in combination with an Iowa Black quencher (e.g., IABKFQ), and/or TAO™ in combination with an Iowa Black quencher (e.g., IABKFQSp).

Illustrative embodiment 31 is the method of any preceding or subsequent illustrative method embodiment for using any of the compositions and/or kits and/or systems of any of the preceding or subsequent composition and/or kit and/or system embodiments.

Illustrative embodiment 32 is a composition for performing any of the preceding or subsequent method embodiments or for using any of the kits and/or systems of any of the preceding or subsequent kit and/or system embodiments.

Illustrative embodiment 33 is the composition of any preceding or subsequent illustrative composition embodiment, comprising at least one primer and/or probe having the sequence of SEQ ID NOs: 1-17.

Illustrative embodiment 34 is a kit for performing any of the preceding or subsequent method embodiments or using any of the compositions and/or systems of any of the preceding or subsequent composition and/or system embodiments.

Illustrative embodiment 35 is the kit of any preceding or subsequent illustrative kit embodiment, comprising at least one primer and/or probe having the sequence of SEQ ID NOs: 1-17.

Illustrative embodiment 36 is the kit of any preceding or subsequent illustrative kit embodiment, comprising instructions for use.

Illustrative embodiment 37 is a system for performing any of the preceding or subsequent method embodiments or using any of the compositions and/or kits of any of the preceding or subsequent composition and/or kit embodiments.

Illustrative embodiment 38 is the system of any preceding or subsequent illustrative system embodiment, further comprising a component and/or station for generating virus-specific cDNA and/or performing RT-PCR using primers and/or probes specific for at least two distinct respiratory viruses and/or a station and/or component for determining the absence and/or presence and/or amount of the at least two distinct respiratory viruses in the sample.

Illustrative embodiment 39 is the system of any preceding or subsequent illustrative system embodiment, further comprising a station and/or component for isolating RNA from a sample from a subject.

Illustrative embodiment 40 is the system of any preceding or subsequent illustrative system embodiment, wherein the at least two distinct respiratory viruses comprise at least two, or at least three, or all four of SARS-CoV-2, influenza A (Flu A), influenza B (Flu B), or Respiratory Syncytial Virus (RSV).

Illustrative embodiment 41 is the system of any preceding or subsequent illustrative system embodiment, wherein the RT-PCR comprises real-time RT-PCR.

Illustrative embodiment 42 is the system of any preceding or subsequent illustrative system embodiment, wherein the RT-PCR comprises multiplex RT-PCR.

Illustrative embodiment 43 is the system of any preceding or subsequent illustrative system embodiment, wherein the sample is self-collected by the subject.

Illustrative embodiment 44 is the system of any preceding or subsequent illustrative system embodiment, wherein any of the stations and/or components may be automated or controlled by a computer.

Illustrative embodiment 45 is the system of any preceding or subsequent illustrative system embodiment, further comprising one or more data processors, and a non-transitory computer readable storage medium containing instructions which, when executed on the one or more data processors, cause the one or more data processors to perform actions to direct at least one of the steps of obtaining a sample from the subject; isolating RNA from the sample; performing RT-PCR using primers and/or probes specific for at least two of SARS-CoV-2, Influenza A, Influenza B and/or RSV, and determining the presence or absence of the at least two of SARS-CoV-2, Influenza A, Influenza B and/or RSV nucleic acid in the sample.

Illustrative embodiment 46 is a computer-program product tangibly embodied in a non-transitory machine-readable storage medium, including instructions configured to perform any of the preceding or subsequent method embodiments or use any of the compositions of any of the preceding or subsequent composition embodiments or use any of the kits of any of the preceding or subsequent kit embodiments or to run any of the components and/or stations of any of the preceding or subsequent composition embodiments.

Illustrative embodiment 47 is the computer-program product of any preceding or subsequent illustrative computer-program embodiment, including instructions configured to cause one or more data processors to perform actions to direct at least one of the steps of obtaining a sample from the subject; isolating RNA from the sample; performing RT-PCR using primers and/or probes specific for at least two of SARS-CoV-2, Influenza A, Influenza B and/or RSV, and determining the presence and/or absence and/or amount of the at least two of SARS-CoV-2, Influenza A, Influenza B and/or RSV nucleic acid in the sample.

Illustrative embodiment 48 is the computer-program product of any preceding or subsequent illustrative computer-program embodiment, wherein the RT-PCR is real-time (quantitative) RT-PCR.

Illustrative embodiment 49 is the computer-program product of any preceding or subsequent illustrative computer-program embodiment, wherein the RT-PCR comprises multiplex RT-PCR.

That which is claimed is:

1. A method to determine whether a sample from a subject contains an infections respiratory virus comprising:
obtaining the sample from a subject;
isolating RNA from the sample;
performing reverse transcriptase polymerase chain reaction (RT-PCR) amplification using primers and probes specific for the at least two distinct respiratory viruses; and
determining the amount and/or presence of at least one of the at least two distinct respiratory viruses in the sample.

2. The method of claim 1, wherein the at least two distinct respiratory viruses comprise at least two of SARS-CoV-2, influenza A (Flu A), influenza B (Flu B), or Respiratory Syncytial Virus (RSV).

3. The method of claim 1, wherein the at least two distinct respiratory viruses comprise at least three of SARS-CoV-2, influenza A (Flu A), influenza B (Flu B), or Respiratory Syncytial Virus (RSV).

4. The method of claim 1, wherein the at least two distinct respiratory viruses comprise each of SARS-CoV-2, influenza A (Flu A), influenza B (Flu B), or Respiratory Syncytial Virus (RSV).

5. The method of claim 1, wherein the sample is an anterior nasal swab or a nasopharyngeal (NP) swab.

6. The method of claim 1, wherein the RT-PCR comprises multiplex RT-PCR.

7. The method of claim 1, wherein the sample is self-collected by the subject.

8. The method of claim 1, wherein the RT-PCR comprises real-time RT-PCR.

9. The method of claim 1, wherein the primers used for RT-PCR amplify a SARS-CoV-2 nucleocapsid (N) gene nucleic acid.

10. The method of claim 1, wherein the primers used for RT-PCR amplify an influenza A virus matrix 1 (M1) gene nucleic acid.

11. The method of claim 1, wherein the primers used for RT-PCR amplify an influenza B virus nonstructural 2 (NS2) gene nucleic acid.

12. The method of claim 1, wherein the primers used for RT-PCR amplify a Respiratory Syncytial Virus matrix (M) gene nucleic acid.

13. The method of claim 1, wherein the primers and probes used for RT-PCR comprise at least one of SEQ ID NOs: 1-17.

14. The method of claim 1, further comprising amplification of a control gene that is present in the subject but is not present in the at least one of the at least two distinct respiratory viruses.

15. The method of claim 1, wherein determining the amount or presence of at least one of the at least two distinct respiratory viruses in the sample comprises a positive result (+) if the at least one of the at least two distinct respiratory viruses produces a Ct value of ≤40 and/or a negative or not detected result (−) if the at least one of the at least two distinct respiratory viruses produces a Ct value of >40.

16. The method of claim 1, wherein a limit of detection (LoD) for each of SARS-CoV-2, Influenza A, Influenza B and/or RSV is <10 copies per microliter (μL).

17. A system to determine whether a sample from a subject contains an infections respiratory virus comprising:
a station and/or component for isolating RNA from a sample from the subject;
a station and/or component for performing reverse transcriptase polymerase chain reaction (RT-PCR) amplification using primers and probes specific for at least two distinct respiratory viruses; and
a station and/or component for determining the amount and/or presence of at least one of the at least two distinct respiratory viruses in the sample.

18. The system of claim 17, wherein the primers and probes used for RT-PCR comprise at least one of SEQ ID NOs: 1-17.

19. A composition to determine whether a sample from a subject contains an at least one respiratory virus comprising primers and probes comprising at least one of SEQ ID NOs: 1-17.

20. The composition of claim 19, wherein the at least one respiratory virus comprises at least two of SARS-CoV-2, influenza A (Flu A), influenza B (Flu B), or Respiratory Syncytial Virus (RSV).


SEQUENCE LISTING (17 sequences)

SEQ ID NO: 1 - DNA, 25 bp, synthetic construct
  source 1..25: mol_type = other DNA
caagaccaatcytgtcacctctgac

SEQ ID NO: 2 - DNA, 25 bp, synthetic construct
  source 1..25: mol_type = other DNA
caagaccaatyctgtcacctytgac

SEQ ID NO: 3 - DNA, 23 bp, synthetic construct
  source 1..23: mol_type = other DNA
gcattytggacaaavcgtctacg

SEQ ID NO: 4 - DNA, 23 bp, synthetic construct
  source 1..23: mol_type = other DNA
gcattttggataaagcgtctacg

SEQ ID NO: 5 - DNA, 24 bp, synthetic construct
  source 1..24: mol_type = other DNA
tgcagtcctcgctcactgggcacg

SEQ ID NO: 6 - DNA, 22 bp, synthetic construct
  source 1..22: mol_type = other DNA
tcctcaaytcactcttcgagcg

SEQ ID NO: 7 - DNA, 21 bp, synthetic construct
  source 1..21: mol_type = other DNA
cggtgctcttgaccaaattgg

SEQ ID NO: 8 - DNA, 27 bp, synthetic construct
  source 1..27: mol_type = other DNA
ccaattcgagcagctgaaactgcggtg

SEQ ID NO: 9 - DNA, 24 bp, synthetic construct
  source 1..24: mol_type = other DNA
ggcaaatatggaaacatacgtgaa

SEQ ID NO: 10 - DNA, 28 bp, synthetic construct
  source 1..28: mol_type = other DNA
tctttttctaggacattgtaytgaacag

SEQ ID NO: 11 - DNA, 27 bp, synthetic construct
  source 1..27: mol_type = other DNA
ctgtgtatgtggagccttcgtgaagct

SEQ ID NO: 12 - DNA, 23 bp, synthetic construct
  source 1..23: mol_type = other DNA
ctgcagatttggatgatttctcc

SEQ ID NO: 13 - DNA, 25 bp, synthetic construct
  source 1..25: mol_type = other DNA
ccttgtgtggtctgcatgagtttag

SEQ ID NO: 14 - DNA, 30 bp, synthetic construct
  source 1..30: mol_type = other DNA
attgcaacaatccatgagcagtgctgactc

SEQ ID NO: 15 - DNA, 19 bp, synthetic construct
  source 1..19: mol_type = other DNA
agatttggacctgcgagcg

SEQ ID NO: 16 - DNA, 20 bp, synthetic construct
  source 1..20: mol_type = other DNA
gagcggctgtctccacaagt

SEQ ID NO: 17 - DNA, 23 bp, synthetic construct
  source 1..23: mol_type = other DNA
ttctgacctgaaggctctgcgcg